(19) United States
(12) Patent Application Publication
(10) Pub. No.: US 2026/0207756 A1
(43) Pub. Date: Jul. 23, 2026

(54) MEDICAL USE OF FUNCTIONALIZED POLYMER

(71) Applicants: MEXBRAIN, VILLEURBANNE (FR); UNIVERSITE CLAUDE BERNARD LYON 1, VILLEURBANNE (FR); INSTITUT GUSTAVE ROUSSY, VILLEJUIF (FR); CENTRE NATIONAL DE LA RECHERCHE SCIENTIFIQUE, PARIS 16 (FR)

(72) Inventors: Daphné MOREL, SAINT PIERRE DE CHANDIEU (FR); Eric DEUTSCH, PARIS (FR); Olivier TILLEMENT, FONTAINES SAINT-MARTIN (FR); François LUX, VILLEURBANNE (FR); Laurent DAVID, LYON (FR); Fabien ROSSETTI, VILLEURBANNE (FR); Arthur DURAND, CLERLANDE (FR); Jordyn HOWARD, BUFORD, GA (US); Elise ROSSON, VILLARS LES DOMBES (FR)

(73) Assignees: MEXBRAIN, VILLEURBANNE (FR); UNIVERSITE CLAUDE BERNARD LYON 1, VILLEURBANNE (FR); INSTITUT GUSTAVE ROUSSY, VILLEJUIF (FR); CENTRE NATIONAL DE LA RECHERCHE SCIENTIFIQUE, PARIS 16 (FR)

(21) Appl. No.: 19/104,121

(22) PCT Filed: Aug. 17, 2023
(86) PCT No.: PCT/EP2023/072670

§ 371 Date: Feb. 14, 2025

(30) Foreign Application Priority Data
Aug. 18, 2022 (EP) 22306238.1

(51) Int. Cl. A61K 47/61; A61K 9/00

(52) U.S. Cl. CPC A61K 47/61; A61K 9/0053

(57) ABSTRACT

The present disclosure relates to a functionalized polymer, for use in treating a disease correlated with heavy metal-induced toxicity or heavy metal-induced carcinogenicity in a subject in need thereof, wherein said functionalized polymer is soluble in aqueous solution, has a weight average molecular mass between 30 kDa and 5000 kDa, and wherein a part of the monomeric units are functionalized with a chelating moiety, wherein therapeutically efficient amount of said functionalized polymer is administered orally to the subject.

### FIELD OF THE INVENTION

The present invention relates to medical use of a functionalized polymer. In particular, the invention relates to functionalized polymer for use in treating a disease correlated with heavy metal-induced toxicity or heavy metal-induced carcinogenicity in a subject in need thereof, wherein said functionalized polymer is soluble in aqueous solution, has a weight average molecular mass between 30 kDa and 5000 kDa, and wherein a part of the monomeric units are functionalized with a chelating moiety, wherein therapeutically efficient amount of said functionalized polymer is administered orally to the subject.

### BACKGROUND

Ubiquitous heavy metals are found within our environment as consequences of the human industrialization of our world (waste disposal, pollution from factories, nuclear reactions, etc.) which humans are inevitably exposed due to polluted air, water, and foodstuff (animals, crops, etc.). Trace levels of lead and cadmium are found within the blood of humans around the world and intake from foodstuff has been directly associated with many serious pathologies including cancer and chronic kidney disease (CKD) (Satarug, S.; Vesey, D. A.; Gobe, G. C. Health Risk Assessment of Dietary Cadmium Intake: Do Current Guidelines Indicate How Much Is Safe? Environ Health Perspect 2017, 125 (3), 284-288; Satarug, S.; C. Gobe, G.; A. Vesey, D.; Phelps, K. R. Cadmium and Lead Exposure, Nephrotoxicity, and Mortality. Toxics 2020, 8 (4), 86; European Food Safety Authority. Cadmium Dietary Exposure in the European Population. EFSA Journal No. 2012; 10(1):2551; Boon, P. E.; Pustjens, A. M.; te Biesebeek, J. D.; Brust, G. M. H.; Castenmiller, J. J. M. Dietary Intake and Risk Assessment of Elements for 1- and 2-Year-Old Children in the Netherlands. Food and Chemical Toxicology 2022, 161, 112810).

High blood concentrations of heavy metals, especially lead and cadmium, are most common in people who have been exposed to high concentrations through work-related exposures in mines and factories. However, even low exposure can lead to a significant blood concentration over time. Once these heavy metals are integrated within the body, the majority is eliminated via natural elimination pathways, but a small portion is absorbed into the bloodstream and accumulated with the organs (liver, kidneys, bones) on a long-term scale. This longer-term exposure has been identified in various regions due to contaminated foodstuff and water, especially within children. Equally as concerning is the vicious cycle of heavy metals existing in the aging population or subjects like certain pathologies, like chronic kidney disease (CKD). These populations experience a reduced efficiency of natural elimination pathways, therefore accumulating heavy metals in larger quantities within these systems and more toxicity.

These metals, along with other heavy metals, have been associated to a wide variety of pathologies, including but not limited to, neurodegeneration and neurodegenerative diseases like Parkinson's disease, cardiovascular disease, kidney failure and chronic kidney disease (CKD), stunted fertility in men, miscarriages, and stunted growth in children (Reuben, A. Childhood Lead Exposure and Adult Neurodegenerative Disease. JAD 2018, 64 (1), 17-42; Kumar, A.; Kumar, A.; M. M.s., C.-P.; Chaturvedi, A. K.; Shabnam, A. A.; Subrahmanyam, G.; Mondal, R.; Gupta, D. K.; Malyan, S. K.; Kumar, S. S.; A. Khan, S.; Yadav, K. K. Lead Toxicity: Health Hazards, Influence on Food Chain, and Sustainable Remediation Approaches. International Journal of Environmental Research and Public Health 2020, 17 (7), 2179; Leggett, R. W. An Age-Specific Kinetic Model of Lead Metabolism in Humans. Environ Health Perspect 1993, 101 (7), 598-616; Rossi, E. Low Level Environmental Lead Exposure—A Continuing Challenge. Clin Biochem Rev 2008, 29 (2), 63-70).

On a cellular level, these metals use their strong affinities for sulfur, nitrogen, and oxygen to bind to and inhibit a wide range of enzymes, proteins, cellular signaling pathways, while increasing the concentration of reactive oxygen species (ROS) (Chen, P.; Bornhorst, J.; Diana Neely, M.; Avila, D. S. Mechanisms and Disease Pathogenesis Underlying Metal-Induced Oxidative Stress. Oxidative Medicine and Cellular Longevity 2018, 2018, 1-3; Ercal, N.; Gurer-Orhan, H.; Aykin-Burns, N. Toxic Metals and Oxidative Stress Part 1: Mechanisms Involved in Metal Induce Oxidative Damage. Current Topics in Medicinal Chemistry. 1st ed. 2001, pp 529-539; Flora, S. J. S.; Mittal, M.; Mehta, A. Heavy Metal Induced Oxidative Stress & Its Possible Reversal by Chelation Therapy. INDIAN J MED RES 2008, 24). On a global scale, these metals are highly nephrotoxic, as they have been directly associated with a decreased glomerulus filtration rate (GFR), and exhibit large organ accumulation after absorption within the bloodstream (Reyes, J. L.; Molina-Jijón, E.; Rodriguez-Munoz, R.; Bautista-Garcia, P.; Debray-Garcia, Y.; Namorado, M. del C. Tight Junction Proteins and Oxidative Stress in Heavy Metals-Induced Nephrotoxicity. BioMed Research International 2013, 2013, 1-14; Johri, N.; Jacquillet, G.; Unwin, R. Heavy Metal Poisoning: The Effects of Cadmium on the Kidney. Biometals: an international journal on the role of metal ions in biology, biochemistry, and medicine 2010, 23, 783-792; Lentini, P.; Zanoli, L.; Granata, A.; Signorelli, S. S.; Castellino, P.; Dellaquila, R. Kidney and Heavy Metals—The Role of Environmental Exposure. Molecular Medicine Reports 2017, 15 (5), 3413-3419). Accumulation within the bones can re-introduce these metals into the bloodstream during osteoporosis and similar pathologies related to aging anatomical systems.

Current therapies for acute heavy metal poisoning (>200 μg/L) includes the administration of chelating moieties, also referred to as chelators, like EDTA and DMSA. However, such therapies are not available to lower blood levels (<100 μg/L) due to inefficiency. Trace levels of lead (<50 μg/L) and cadmium (<1 μg/L) have been associated with insidious symptoms in humans, although these values are considered “acceptable” and do not require therapeutic strategies as defined by WHO and CDC. The only therapeutic strategy proposed for this population is to distance themselves from the source of exposure (ABLES—Reference Blood Lead Levels (BLLs) for Adults in the U. S.|NIOSH|CDC. https://www.cdc.gov/niosh/topics/ables/ReferenceBloodLevelsforAdults.html(accessed 2022 May 23); Center for Disease Control (CDC). Blood Lead Reference Value|Lead|CDC. https://www.cdc.gov/nceh/lead/data/blood-lead-reference-value.htm (accessed 2022 May. 19); World Health Organization; Regional Office for Europe; Joint WHO/Convention Task Force on the Health Aspects of Air Pollution. Health Risks of Heavy Metals from Long-Range Transboundary Air Pollution; World Health Organization Regional Office Europe: Copenhagen, 2007).

Accordingly, distancing one's self from the source of exposure is extremely difficult when it comes from foodstuff and water.

WO2019/122790 discloses a medical device introducible into the body for the maintenance of metal homeostasis for therapeutic purposes comprising a chelating moiety for extracting metals.

WO2022/023677 describes a statistical polysaccharide with a weight-average molecular weight of between 100 kDa and 1000 kDa and its use in a dialysis process in order to capture at least one metal, in an MRI imaging process, in a brachytherapy process or in a process for marking foodstuffs to prevent forgeries.

It remains therefore a challenge to design a safe and efficient treatment which meets this clinical need of treating diseases correlated with heavy metal-induced toxicity or carcinogenicity in a subject in need thereof.

### SUMMARY OF THE INVENTION

A first aspect of the disclosure relates to a functionalized polymer, for use in treating a disease correlated with heavy metal-induced toxicity or heavy metal-induced carcinogenicity in a subject in need thereof, wherein said functionalized polymer is soluble in aqueous solution, has a weight average molecular mass between 30 kDa and 5000 kDa, and wherein a part of the monomeric units are functionalized with a chelating moiety, wherein therapeutically efficient amount of said functionalized polymer is administered orally to the subject.

A second aspect of the disclosure relates to an oral formulation comprising a functionalized polymer as defined above, and one or more pharmaceutically acceptable excipients, wherein the unit dose of said functionalized polymer is of between 0.1 mg and 500 mg, preferably between 1 mg and 100 mg, more preferably between 2 mg and 10 mg, even more preferably about 5 mg.

### DETAILED DESCRIPTION OF THE INVENTION

### Definitions

In the following, terms as used herein are defined in their meaning.

The term “about” or “ca.” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight by weight percent (w/w %).

As used herein, unless specified otherwise, the term “treating” or “treatment”, denotes reversing, alleviating, inhibiting the progress of, or preventing, diminishing the disorder or condition to which such term applies, or reversing, alleviating, inhibiting the progress of, or preventing one or more symptoms of the disorder or condition to which such term applies. More preferably, in the context of the present invention, “treating” may include a diminution of heavy metals in the blood of a subject or preventing the increase of certain heavy metal in the blood due to passage of heavy metals from water or alimentation to blood circulation.

As used herein, the term “preventing” means at least significantly limiting the uptake of certain heavy metal in the blood of the subject. For example, significantly limiting means at least reducing the uptake of 10%, 20%, 30%, 40%, 50%, 60%, 70%, 80%, 90% or even 100% of heavy metals in a subject exposed to heavy metals compared to a subject not treated with said functionalized polymer of the disclosure and exposed to heavy metals.

As used herein, the term “heavy metal,” “heavy metal species,” and “heavy metal ion” are used interchangeably to designate the atoms and cations of those metals which are toxic to humans. These toxic metals serve absolutely no function within biological systems, and even in trace levels pose threats to the wellbeing and function of the system. Such threats include enzyme and protein inhibition, oxidation of cellular membrane and degradation of signaling pathways, DNA repair inhibition and promotion of DNA degradation, apoptosis, mutagenesis, etc which therefore lead to cardiovascular disease, neurodegeneration and cognitive difficulties, kidney dysfunction, cancers, and mortality. For example heavy metals may be selected from the group consisting of lead (Pb), cadmium (Cd), arsenic (As), chromium (Cr), mercury (Hg), cobalt (Co), aluminium (AI), antinomy (Sb), barium (Ba), bismuth (Bi), gallium (Ga), germanium (Ge), gold (Au), indium (In), nickel (Ni), platinum (Pt), silver (Ag), strontium (Sr), tellurium (Te), thallium (Tl), tin (Sn), titanium (Ti), vanadium (V), plutonium (Pu), cesium (Cs), cerium (Ce), zirconium (Zr), ruthenium (Ru), technetium (Tc), radium (Ra), thorium (Th), americium (Am), iridium (Ir), californium (Cf), polonium (Po) and uranium (U), or mixtures thereof, preferably selected from the group consisting of lead, cadmium, arsenic, chromium and mercury, more preferably lead or cadmium. In a specific embodiment, heavy metals may be radioactive and may be selected from the group consisting of Co(60), U(232, 233, 234, 235, 236 or even 238), Pu (238, 239, 240, 241), Sr(90), Cs(135, 137), Ce(144), Zr(93, 95), Ru(106), Tc(99), Sn(126), Am(241), Ir(192), Po(210), Ra(226), Pu(238), Am(241), Cf(252).

As used herein, the term “chelator” or “chelating agent” or “chelating moiety” is used to define a chemical structure or moiety which exhibits a relatively high affinity for certain elements and displays at least two coordination sites. The affinity is such that the chelator is able to chelate the metal in a semi-permanent sense and can remove them from any affinity that they show with biomolecules. A chelator may function primarily to “neutralize” heavy metals by maintaining their chelation to these metals and preventing their reactions and interactions with other biomaterials. A chelator may exhibit a pincer-type structure or moiety with two or more opposed portions formed by chemical groups that have negative charges within biological environments (sulfhydryl groups, ketone groups, carboxy groups, hydroxyl groups, etc.) or neutral charge (amino group). These groups are spaced accordingly to allow for the comfortable accommodation of the metal ion within their structure.

The chelating moiety in the context of the present disclosure may be
DOTA: 1,4,7,10-tetraazacyclododecane-N,N′,N″,N′″-teracetic acid;
NOTA: 1,4,7-triazacyclononane-1,4, 7-triacetic acid;
NODAGA: 1,4,7-triazacyclononane-1-glutaric-4,7-diacetic acid;
DOTAGA:2-(4,7,10-tris(carboxymethyl)-1,4,7, 10-tetraazacyclododecan-1-yl)pentanedioicacid;
DOTAM: 1,4,7,10-tetrakis(carbamoylmethyl)-1,4,7,10-tetraazacyclodecane;
NOTAM: 1,4,7-tetrakis(carbamoylmethyl)-1, 4,7-triazacyclononane;
DOTP: 1,4,7,10-tetraazacyclododecane 1,4,7,10-tetrakis(methylene phosphonate;
NOTP: 1,4,7-tetrakis(methylene phosphonate)-1, 4,7-triazacyclononane;
TETA: 1,4,8,11-tetraazacyclotetradecane-N,N′,N″,N′″-teracetic acid;
TETAM: 1,4,8, 11-tetraazacyclotetradecane-N,N′,N,N′″-tetrakis(carbamoyl methyl);
DTPA: diethylene triaminopentaacetic acid;
Bz-DFO: benzyl deferoxamine;
DFO: deferoxamine.

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease. In specific embodiment, such therapeutically efficient amount is an amount sufficient to prevent the uptake of said heavy metal in the body of a subject.

As used herein, the term “independently” for the Rc moiety i.e. the chelating moiety, means that each Rc of the functionalized statistic chitosan of formula (I) is a chelating moiety that may be different from one to another, or may be identical. For example, each Rc is identical i.e., there is one type of Rc throughout the functionalized statistic chitosan, or there may be more than one type of Rc throughout the functionalized statistic chitosan i.e. two, three, four, five or even n different Rc, n being an integer.

The term “pharmaceutically acceptable excipients”, as used herein, refers to a non-active substance that is added alongside the drug substance, and is part of the formulation mixture. Pharmaceutically acceptable excipients are for example fillers, solvents, diluents, carriers, auxiliaries, distributing and sensing agents, delivery agents, such as preserving agents, disintegrants, moisteners, emulsifiers, suspending agents, thickeners, sweeteners, flavouring agents, aromatizing agents, antibacterial agents, fungicides, lubricants, and prolonged delivery controllers, antioxidants, glidants. The choice and suitable proportions of them are depended on the nature and way of administration and dosage.

As used herein “combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The terms “patient”, “subject”, “individual”, and the like, are used interchangeably herein, and refer to a human. In some embodiments, the patient, subject or individual in need of treatment includes those who already have the disease, condition, or disorder, i.e. disease correlated with heavy metal-induced toxicity or heavy metal-induced carcinogenicity.

### The Functionalized Polymer for Use According to the Present Disclosure

In a first aspect of the disclosure relates to a functionalized polymer, for use in treating a disease correlated with heavy metal-induced toxicity or heavy metal-induced carcinogenicity in a subject in need thereof, wherein said functionalized polymer is soluble in aqueous solution, has a weight average molecular mass between 30 kDa and 5000 kDa, and wherein a part of the monomeric units are functionalized with a chelating moiety, wherein therapeutically efficient amount of said functionalized polymer is administered orally to the subject.

In certain embodiment, the functionalized polymer has a weight average molecular mass between 100 kDa and 1000 kDa, preferably between 150 kDa and 750 kDa, more preferably between 200 kDa and 600 kDa, even more preferably between 250 kDa and 400 kDa, and even more preferably about 300 kDa.

In another embodiment, the functionalized polymer has a weight average molecular mass between 30 kDa and 100 kDa, preferably between 32 kDa and 80 kDa, more preferably between 34 kDa and 60 kDa, even more preferably between 35 kDa and 50 kDa, and even more preferably about 40 kDa.

In an embodiment, said functionalized polymer comprises at least 1 w % of chelating moiety, for example between 1 w % and 40 w %, preferably between 5 w % and 30 w %, more preferably between 10 w % and 25 w %, even more preferably about 10 w % or 17 w % or 22 w %.

Said chelating moiety enables chelation of one or more metals. Each of the chelating moiety may comprise two or more coordination sites. Preferably, the coordination site is a nitrogen or oxygen atom. Advantageously, each of the chelating moiety comprises between 4 and 8 coordination sites, more preferably between 6 and 8 coordination sites and even more preferably each of the chelating moiety comprises 6 coordination sites.

As used herein, the term “coordination site” refers to a single function capable of complexing a metal. For example, an amine function represents a coordination site by the formation of a dative bond between the nitrogen atom and the metal, and a hydroxamic acid function also represents a coordination site by the formation of a dative bond between the oxygen of the carbonyl unit and by a covalent bond with the oxygen of the N-oxide unit the coordination site thus forming a five-membered ring.

In an embodiment, each chelating moiety is selected from the group consisting of DOTA (1,4,7,10-tetraazacyclododecane-N,N′,N″,N′″-teracetic acid), NOTA (1,4,7-triazacyclononane-1,4, 7-triacetic acid), NODAGA (1,4,7-triazacyclononane-1-glutaric-4,7-diacetic acid), DOTAGA (2-(4,7,10-tris(carboxymethyl)-1,4,7, 10-tetraazacyclododecan-1-yl)pentanedioic acid), DOTAM (1,4,7,10-tetrakis(carbamoylmethyl)-1,4,7,10-tetraazacyclodecane), NOTAM (1,4,7-tetrakis(carbamoylmethyl)-1, 4,7-triazacyclononane), DOTP (1,4,7,10-tetraazacyclododecane 1,4,7,10-tetrakis(methylene phosphonate), NOTP (1,4,7-tetrakis(methylene phosphonate)-1, 4,7-triazacyclononane), TETA (1,4,8,11-tetraazacyclotetradecane-N, N′,N″,N′″-teracetic acid), TETAM (1,4,8, 11-tetraazacyclotetradecane-N, N′,N″,N′″-tetrakis(carbamoyl methyl), DTPA (diethylene triaminopentaacetic acid) Bz-DFO (benzyl deferoxamine) and DFO (deferoxamine) and mixtures thereof, preferably from the group consisting of DOTAGA, Bz-DFO, DFO, DOTAM and DTPA, and more preferably the chelating moiety is DOTAGA, or DOTAM, or DTPA, or a mixture of DOTAGA and Bz-DFO, or a mixture of DOTAGA and NODAGA, or a mixture of DOTAGA and DTPA, or a mixture of DOTAGA and DOTAM.

In an embodiment, the chelating moiety is selected from the group consisting of:

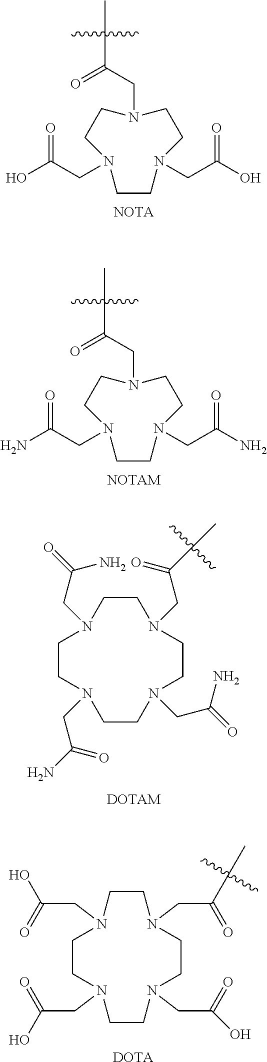

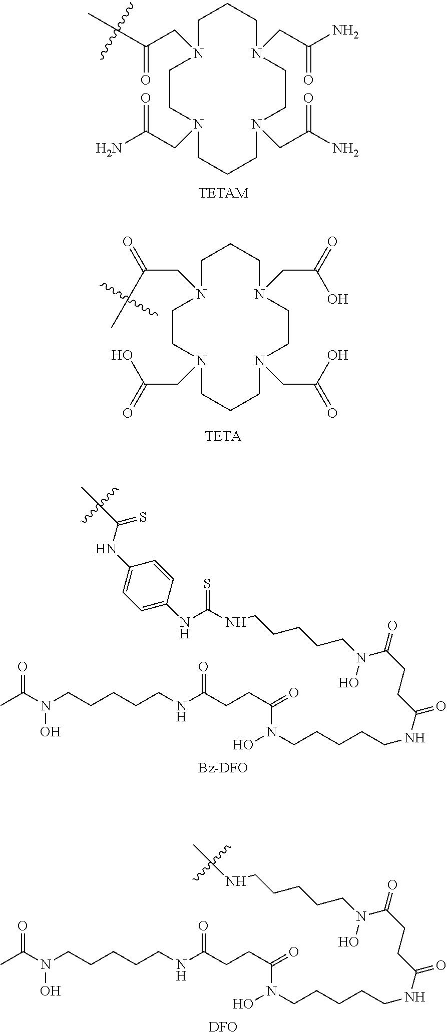

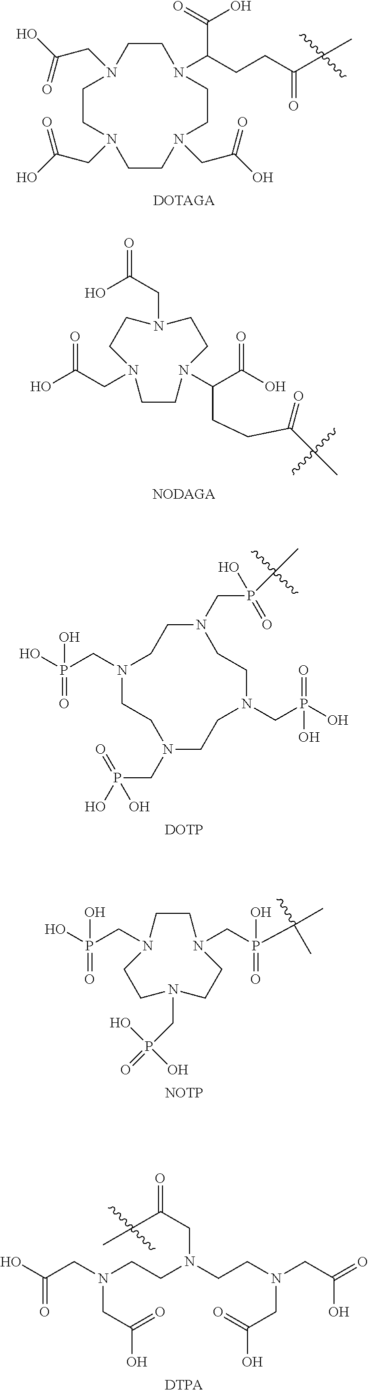

In specific embodiments, said heavy metal that is chelated by a chelating moiety or mixtures thereof is selected from the group consisting of lead (Pb), cadmium (Cd), arsenic (As), chromium (Cr), mercury (Hg), cobalt (Co), aluminium (AI), antinomy (Sb), barium (Ba), bismuth (Bi), gallium (Ga), germanium (Ge), gold (Au), indium (In), nickel (Ni), platinum (Pt), silver (Ag), strontium (Sr), tellurium (Te), thallium (Tl), tin (Sn), titanium (Ti), vanadium (V), plutonium (Pu), cesium (Cs), cerium (Ce), zirconium (Zr), ruthenium (Ru), technetium (Tc), radium (Ra), thorium (Th), americium (Am), iridium (Ir), californium (Cf), polonium (Po) and uranium (U), or mixtures thereof, preferably selected from the group consisting of lead, cadmium, arsenic, chromium and mercury, more preferably lead or cadmium.

In a specific embodiment, said heavy metal is radioactive and is for example selected from the group consisting of Co(60), U(232, 233, 234, 235, 236 or even 238), Pu (238, 239, 240, 241), Sr(90), Cs(135, 137), Ce(144), Zr(93, 95), Ru(106), Tc(99), Sn(126), Am(241), Ir(192), Po(210), Ra(226), Pu(238), Am(241), Cf(252).

The functionalized polymer may be selected from the group consisting of polysaccharides, polyethylene glycol (PEG), polyvinyl alcohol (PVA), polyacrylic acid (PAA), poly allylamine (PAH), wherein a part of the monomeric units is functionalized with a chelating moiety.

In specific embodiment, polysaccharides may be chosen among digestible polysaccharides such as starch, or non-digestible polysaccharides such as chitosan, cellulose, chitin, β-glucan, xylan, pectin, mucilage, gums, lignin, galactan, agar, fructan, fucoidans galactoglucans, sulfated polysaccharides or mixtures thereof, preferably among chitosan. In preferred embodiment, polysaccharides are chosen among non-digestible polysaccharides. Indeed, non-digestible polysaccharides possess beta glycosidic bonds which will be less easily digested compared to digestible polysaccharides which possess alpha glycosidic bonds that are digested more quickly via the alpha amylases present in the digestive tract.

In an embodiment, the functionalized polymer is polyethylene glycol (PEG), preferably with DTPA or DOTAM or mixtures thereof as chelating moiety and more preferably wherein the polyethylene glycol has a weight average molecular mass between 30 kDa and 100 kDa, preferably between 32 kDa and 80 kDa, more preferably between 34 kDa and 60 kDa, even more preferably between 35 kDa and 50 kDa, and even more preferably about 40 kDa.

In specific embodiments, the functionalized polymer of the present disclosure is selected among polymers which do not reach the bloodstream after oral administration. As used herein, a polymer is considered as not reaching the bloodstream after oral administration when less than 5% of the polymer, preferably less than 3%, more preferably less than 1% is found in the blood after oral administration, typically as determined in an animal assay as described in the examples, i.e. fluorescence study within red filter for gadolinium quantification using ICPMS.

Indeed, without wishing to be bound by any theory, the inventors believe that by remaining in the gastrointestinal tract, the polymer advantageously acts locally by chelating a portion of the heavy metals present in the water and/or foodstuff and/or air pollution, for example from a contamination, a pollution, an accident, a combat zone such as dirty bomb, prior to their assimilation and therefore, the polymer prevents their passage through the intestinal barrier. The fact that the polymer does not pass the intestinal barrier therefore allows a local action in the gastro-intestinal tract and avoids the common adverse effects of the presence of a chelating moiety in the systemic compartment as observed in prior art treatments.

In an embodiment, at least 90% of said polymer, preferably at least 95%, more preferably at least 99%, is eliminated in faeces within 7 days after oral administration to said subject.

In another embodiment, the polymer for use according to the present disclosure is selected among the polymers which are poorly degraded in the gastrointestinal tract in such a way that its weight average molecular mass does not substantially vary after its passage in the gastrointestinal tract.

Preferred embodiments of the polymer: the functionalized PEG In a specific embodiment, the functionalized polymer is polyethylene glycol (PEG), more preferably an 8-arm PEG amine according to the following formula (V)

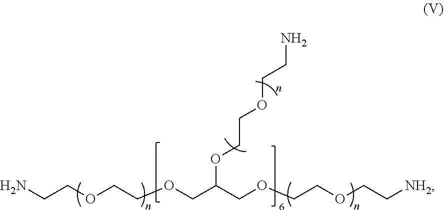

wherein n is between 100 and 120, more preferably between 105 and 115, even more preferably between 109 and 113, even more preferably about 111.

In an embodiment, the 8-arm PEG of formula V is a functionalized PEG, PEG-DTPA, of formula (VI):

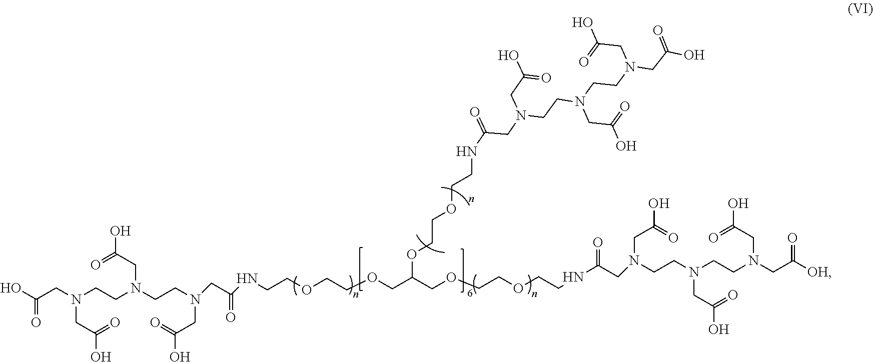

wherein n is between 100 and 120, more preferably between 105 and 115, even more preferably between 109 and 113, even more preferably about 111.

In another embodiment, the 8-arm PEG of formula V is a functionalized PEG, PEG-DOTAM, of formula (VII):

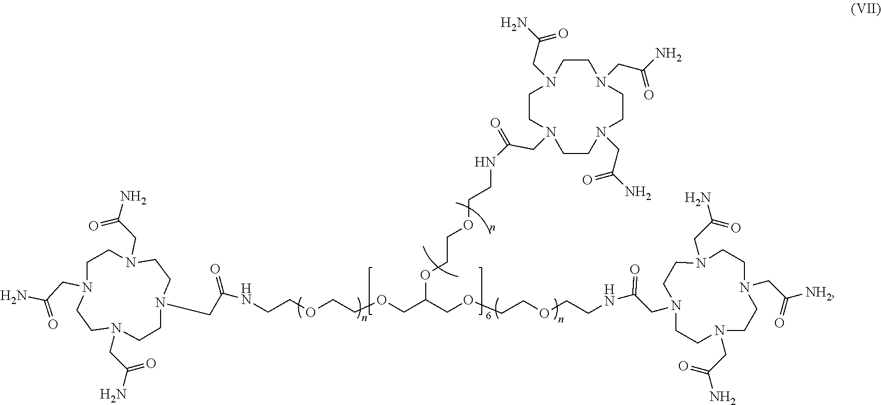

wherein n is between 100 and 120, more preferably between 105 and 115, even more preferably between 109 and 113, even more preferably about 111.

### Preferred Embodiments of the Polymer: The Functionalized Statistic Chitosan

In a specific embodiment, the functionalized polymer is chitosan, preferably a functionalized chitosan having a statistic macromolecular structure of weight average molecular mass between 100 kDa and 1000 kDa and of formula (I):

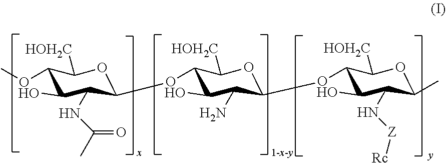

wherein
each Rc is the chelating moiety,
each Z is independently a linker which is a single bond or a hydrocarbon chain containing between 1 and 12 carbon atoms, said hydrocarbon chain is linear or branched and optionally contains one or more unsaturations and being able to contain one or more heteroatoms, preferably chosen from nitrogen, oxygen, sulfur and halogens,
x is between 0.005 and 0.7, preferably between 0.05 and 0.7,
y is between 0.01 and 0.7, preferably between 0.05 and 0.2,
the ratio y/x being greater than or equal to 0.05, preferably greater than or equal to 0.15, and
the sum x+y being greater than or equal to 0.15, preferably greater than or equal to 0.30, more preferably greater than or equal to 0.35.

It is understood that, in the above formula I, more than one Rc group may be present in the functionalized statistic chitosan. These Rc groups may be the same or different from each other. They are all independently selected from the groups carrying a chelating moiety. The same applies to the Z-linkers, several Z-linkers may be present, and they may be identical or different from each other.

In an embodiment, in formula I, x is between 0.005 and 0.6; y is between 0.1 and 0.9; the ratio y/x being greater than 0.16; and the sum x+y being greater than 0.30.

In a specific embodiment, the functionalized statistic chitosan of the present disclosure has a complexation constant of at least 10¹⁵ for a d or f transition element.

In an embodiment, the functionalized statistic chitosan of formula I is a functionalized statistic chitosan of formula (II):

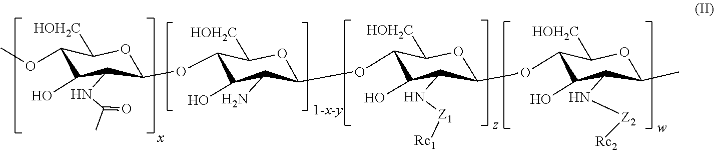

wherein
Rc₁ and Rc₂ are different chelating moieties,
Z₁ and Z₂, identical or different, are linkers which are a single bond or a hydrocarbon chain containing between 1 and 12 carbon atoms, said hydrocarbon chain is linear or branched and optionally contains one or more unsaturations and one or more heteroatoms, preferably chosen from nitrogen, oxygen, sulfur and halogens,
x is between 0.005 and 0.7, preferably between 0.05 and 0.7, and more preferably between 0.2 and 0.6,
y=z+w is between 0.01 and 0.7, preferably between 0.05 and 0.2,
the ratio y/x being greater than or equal to 0.05, preferably greater than or equal to 0.15,
the sum x+y being greater than or equal to 0.15, preferably greater than or equal 0.30, more preferably greater than or equal to 0.35, and
z/y is between 0.5 and 1.

In this embodiment, the functionalized statistic chitosan of formula (II) may comprise either a single type of moiety comprising a chelating moiety, Rc1, when z is equal to 1, or 2 types of moieties comprising a chelating moiety, Rc1 and Rc2, when 0.5≤z<1.

In an embodiment, z/y is between 0.8 and 0.99, Rc₁ moiety is thus in the majority.

In another embodiment, in formula II, x is between 0.005 and 0.6; y is between 0.1 and 0.9; the ratio y/x being greater than 0.3; and z is between 0.5 and 1.

### Rc Moiety (Rc, Rc1 and Rc2)

As used herein, the term “Rc moiety” refers to Rc moiety of formula I, and the terms “Rc1 and Rc2 moiety” refer to Rc₁ and Rc₂ of formula II, when Rc₂ is present. According to the present disclosure, Rc₁ and Rc₂ groups are chelating moieties. In other words, the Rc, Rc₁ and Rc₂ moieties enable chelation of one or more metals by forming a complex.

Each of the Rc, Rc₁ and Rc₂ moiety may comprise two or more coordination sites. Preferably, the coordination site is a nitrogen or oxygen atom. Advantageously, each of the Rc, Rc₁ and Rc₂ moiety comprises between 4 and 8 coordination sites, more preferably between 6 and 8 coordination sites and even more preferably each of the Rc, Rc₁ and Rc₂ moiety comprises 6 coordination sites.

As used herein, the term “coordination site” refers to a single function capable of complexing a metal. For example, an amine function represents a coordination site by the formation of a dative bond between the nitrogen atom and the metal, and a hydroxamic acid function also represents a coordination site by the formation of a dative bond between the oxygen of the carbonyl unit and by a covalent bond with the oxygen of the N-oxide unit the coordination site thus forming a five-membered ring.

In an embodiment, for the functionalized statistic chitosan of formula I, each Rc moiety is independently selected from the group consisting of DOTA (1,4,7,10-tetraazacyclododecane-N,N′,N,N′″-teracetic acid), NOTA (1,4,7-triazacyclononane-1,4, 7-triacetic acid), NODAGA (1,4,7-triazacyclononane-1-glutaric-4,7-diacetic acid), DOTAGA (2-(4,7,10-tris(carboxymethyl)-1,4,7, 10-tetraazacyclododecan-1-yl)pentanedioic acid), DOTAM (1,4,7,10-tetrakis(carbamoylmethyl)-1,4,7,10-tetraazacyclodecane), NOTAM (1,4,7-tetrakis(carbamoylmethyl)-1, 4,7-triazacyclononane), DOTP (1,4,7,10-tetraazacyclododecane 1,4,7,10-tetrakis(methylene phosphonate), NOTP (1,4,7-tetrakis(methylene phosphonate)-1, 4,7-triazacyclononane), TETA (1,4,8,11-tetraazacyclotetradecane-N,N′,N,N′″-teracetic acid), TETAM (1,4,8, 11-tetraazacyclotetradecane-N,N′,N″,N′″-tetrakis(carbamoyl methyl), DTPA (diethylene triaminopentaacetic acid) Bz-DFO (benzyl deferoxamine) and DFO (deferoxamine), preferably from the group consisting of DOTAGA, Bz-DFO, DFO, DOTAM and DTPA, and more preferably the Rc group is DOTAGA.

In another embodiment, for the functionalized statistic chitosan of formula II, Rc1 and Rc2 are independently selected from the group consisting of DOTA, NOTA, NODAGA, DOTAGA, DOTAM, NOTAM, DOTP, NOTP, TETA, TETAM, DTPA, Bz-DFO and DFO, preferably from the group consisting of DOTAGA, Bz-DFO, DFO, DOTAM and DTPA.

In an embodiment, the chelating moiety is selected from the group consisting of:

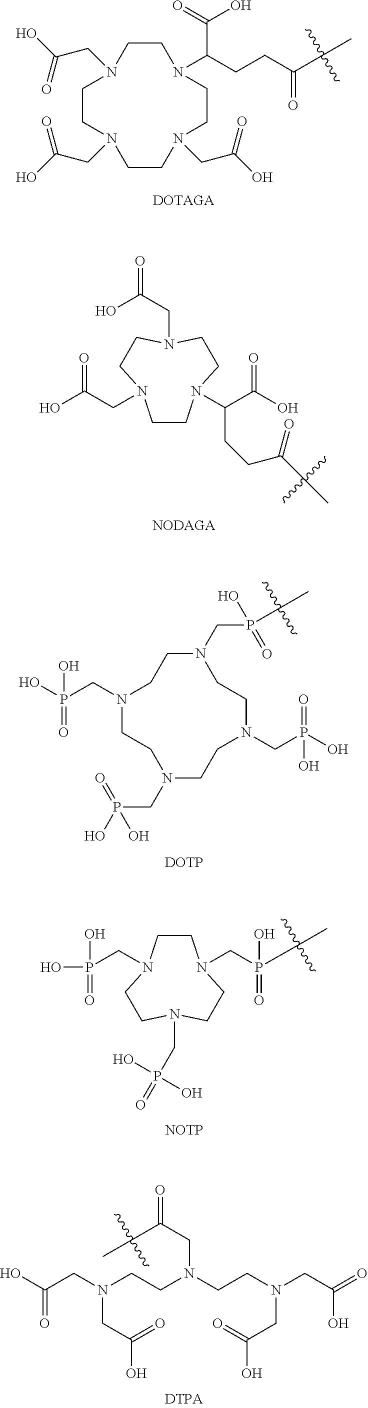

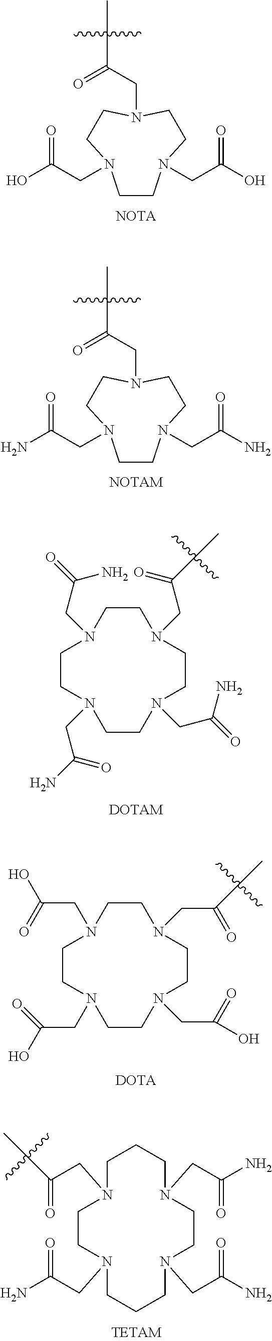

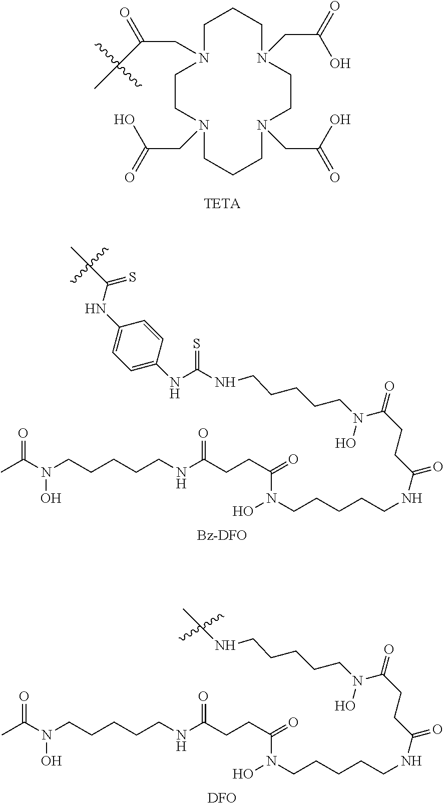 [SMILES: *C(=O)CN1CCCN(CC(=O)O)CCN(CC(=O)O)CCCN(CC(=O)O)CC1 ; *C(=S)Nc1ccc(NC(=S)NCCCCCN(O)C(=O)CCC(=O)NCCCCCN(O)C(=O)CCC(=O)NCCCCCN(O)C(C)=O)cc1 ; *NCCCCCN(O)C(=O)CCC(=O)NCCCCCN(O)C(=O)CCC(=O)NCCCCCN(O)C(C)=O ; "DFO"]

In an embodiment, for the functionalized statistic chitosan of formula I, the group Rc is DOTAGA, and preferably, z/y=1.

In another embodiment, for the functionalized statistic chitosan of formula II, the group Rc1 is DOTAGA and the group Rc2 is Bz-DFO.

### Z Linkers (Z, Z1 and Z2)

As used herein, the term “Z linkers” refers to Z linker of formula I, and the terms “Z₁ and Z₂ linkers” refer to Z₁ and Z₂ linkers of formula II, when the Z₂ binder is present.

The choice of the Z, Z₁ and Z₂ linkers in formula I and II depends essentially on the Rc, Rc₁ and Rc₂ moieties and the metal to be chelated. Indeed, for stearic reasons in particular, the Rc, Rc₁ and Rc₂ moieties may be more or less close to the 6-membered ring of the nitrogen of the glucosamine unit.

In formula I, each Z is independently a linker which is a single bond or a hydrocarbon chain containing between 1 and 12 carbon atoms, said hydrocarbon chain is linear or branched and optionally contains one or more unsaturation and one or more heteroatoms, preferably chosen from nitrogen, oxygen, sulfur and halogens.

In an embodiment, in formula I, each Z is independently selected from the group consisting of: a bond, a linear or branched alkyl chain having between 1 and 12 carbon atoms, and a linear or branched alkenyl chain having between 2 and 12 carbon atoms, said alkyl and alkenyl chains may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR, said alkyl and alkenyl chains may be substituted with one or more groups selected from the group consisting of halogen, —OR′, —COOR′, —SR′, —NR′2, each R′ being independently H or C1-C6 alkyl.

Advantageously, in Formula I, each Z is independently selected from the group consisting of: a bond and a linear or branched alkyl chain having between 1 and 12 carbon atoms, said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In an embodiment each Z is an alkyl chain having between 1 and 12 carbon atoms.

In another embodiment, each Z is a polyethylene glycol (PEG).

Advantageously, in Formula 1l, Z₁ and Z₂ are independently a single bond or a hydrocarbon chain having between 1 and 12 carbon atoms, wherein said chain may be linear or branched and may have one or more unsaturations and may have one or more heteroatoms, preferably selected from nitrogen, oxygen, sulfur, and halogens.

In an embodiment, in formula II, Z₁ and Z₂ are independently selected from the group consisting of: a bond, a linear or branched alkyl chain having between 1 and 12 carbon atoms, and a linear or branched alkenyl chain having between 2 and 12 carbon atoms, said alkyl and alkenyl chains may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR, said alkyl and alkenyl chains may be substituted with one or more groups selected from the group consisting of halogen, —OR′, —COOR′, —SR′, —NR′2, each R′ being independently H or C1-C6 alkyl.

In an embodiment, in Formula II, Z₁ and Z₂ are independently selected from the group consisting of: a bond and a straight or branched alkyl chain having between 1 and 12 carbon atoms, wherein said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In a specific embodiment Z₁ and/or Z₂ is an alkyl chain having between 1 and 12 carbon atoms.

In another specific embodiment, Z₁ and/or Z₂ is a polyethylene glycol (PEG).

### Monomeric Units of the Functionalized Statistic Chitosan

The functionalized statistic chitosan in formulae I of the present disclosure is composed of 3 different monomeric units, namely an N-acetyl glucosamine type A unit, a glucosamine type B unit and a glucosamine type C unit functionalized by a chelating moiety (of the Rc type) linked by a linker (of the Z type) to the nitrogen of the glucosamine.

The functionalized statistic chitosan is statistic polymer. In other words, the sequence of the individual monomer units A, B and type C is random.

The functionalized statistic chitosan in formulae II of the present disclosure is composed of 4 different monomeric units, namely an N-acetyl glucosamine type A unit, a glucosamine type B unit and two glucosamine type C unit, namely C1 and C2, functionalized by a chelating moiety (of the Rc1 type or Rc2 type) linked by a linker (of the Z1 type or Z2 type) to the nitrogen of the glucosamine.

The functionalized statistic chitosan in formulae II is statistic polymer. In other words, the sequence of the individual monomer units A, B, C1 and C2 is random.

In formulae I and II, x represents the proportion of A units and x is between 0.005 and 0.7, preferably between 0.05 and 0.7, more preferably between 0.2 and 0.6, even more preferably x is between 0.25 and 0.4, typically about 0.3. In an embodiment, x is between 0.025 and 0.075, more preferably between 0.04 and 0.06, typically about 0.05.

In formulae I and II, y represents the proportion of C-type units and y is between 0.01 and 0.7, preferably between 0.05 and 0.2. In an embodiment, y is between 0.03 and 0.2, preferably between 0.05 and 0.1, even more preferably between 0.07 and 0.08, typically about 0.072. In another embodiment, y is between 0.05 and 0.3, more preferably between 0.1 and 0.2, typically about 0.15 or 0.12.

In another embodiment, when the Rc1 and Rc2 of formula II are independently selected from the group consisting of DOTA, NOTA, NODAGA, DOTAGA, DOTAM, NOTAM, DOTP, NOTP, TETA, TETAM, DTPA, Bz-DFO and DFO, preferably from the group consisting of DOTAGA, Bz-DFO, DFO, DOTAM and DTPA, and y is between 0.05 and 0.3, more preferably between 0.1 and 0.2, typically about 0.12, Rc1 is between 0.06 and 0.08, typically about 0.07 and Rc2 is between 0.04 and 0.06, typically about 0.05.

The rest of the monomer units in formulae I and II are B units. Thus, in formulae I and II, the proportion of B units is equal to 1-x-y.

According to the disclosure, in formulae I and II, the ratio y/x is greater than or equal to 0.05, preferably greater than or equal to 0.15. Indeed, effectiveness of the functionalized statistic chitosan is determined by the number of chelation sites, which is directly related to the number of metals required, for example, for reducing local inflammation induced by and/or inducing a deregulation of metal homeostasis and for reducing oxidative stress, in a subject in need thereof.

To be administered as a solution to a subject in need thereof while being effective, the functionalized statistic chitosan must be soluble at physiological pH, i.e. pH of between 4.8 and 8. For this purpose, the sum of x+y may be greater than or equal to 0.15, preferably greater than or equal to 0.30, more preferably greater than or equal to 0.35.

The combination of the particular ratio between the number of A units and the number of C units and the sum of the proportion of A units and the proportion of C units makes it possible to obtain adequate chelation and solubility allowing the functionalized statistic chitosan to be used in the treatment of diseases characterized by local inflammation induced by and/or inducing a deregulation of metal homeostasis and increased oxidative stress, in a subject in need thereof.

In accordance with the invention, z/y is between 0.5 and 1. In other words, the C-type units may be exclusively units having Z1 as a linker and Rc1 as a chelating moiety-bearing group.

The functionalized statistic chitosan has a weight average molecular mass between 100 kDa and 1000 kDa, preferably between 150 kDa and 750 kDa, more preferably between 200 kDa and 600 kDa, even more preferably between 250 kDa and 400 kDa, and even more preferably about 300 kDa.

In an embodiment, the functionalized statistic chitosan is selected from the following functionalized statistic chitosan:
a functionalized statistic chitosan of formula II where z/y=1, Rc1 is DOTAGA and Z1 is a bond;
a functionalized statistic chitosan of formula II where z/y=1, Rc1 is DTPA and Z1 is a bond; and
a functionalized statistic chitosan of formula II where 0.5≤z/y<1, Rc1 is DOTAGA and Z1 is a bond, and Rc2 is Bz-DFO and Z2 is selected from the group consisting of: a bond and a straight or branched alkyl chain having between 1 and 12 carbon atoms, wherein said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In an embodiment, the functionalized statistic chitosan has the following formula (III):

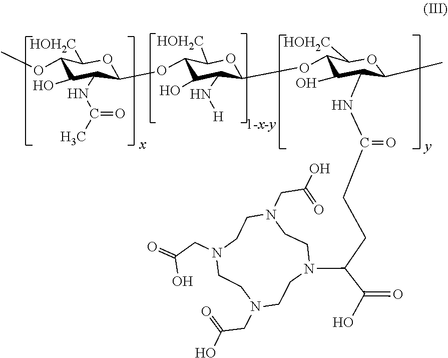

wherein x is between 0.25 and 0.4, typically about 0.3, and y is between 0.05 and 0.2, typically about 0.07.

In an embodiment, the functionalized statistic chitosan is soluble in aqueous solution at physiological pH i.e. pH of between 4.8 and 8 and responds the following: (DS_{DOTAGA}(%)+3.5)*(DA(%)+8)≥150 wherein DS is the degree of substitution of the DOTAGA and DA is the degree of acetylation of the functionalized statistic chitosan.

In another embodiment, the functionalized statistic chitosan has the formula (III), wherein x is between 0.025 and 0.075, more preferably between 0.04 and 0.06, typically about 0.05, and y is between 0.05 and 0.3, more preferably between 0.1 and 0.2, typically about 0.15.

In another embodiment, the functionalized statistic chitosan has the following formula (IV):

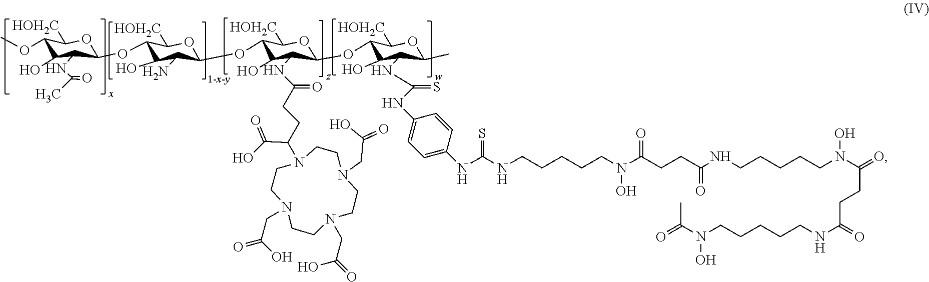

wherein x is between 0.2 and 0.6, more preferably x is between 0.25 and 0.4, typically about 0.3, and y=z+w is between 0.05 and 0.3, more preferably between 0.1 and 0.2, typically about 0.12.

### Synthesis of the Functionalized Polymers of Formula I and II

The compounds of formulae I and II can be synthesized using the methods disclosed in WO 2022/023677 and in the reference Natuzzi, M., Grange, C., Gréa, T. et al. Feasibility Study and Direct Extraction of Endogenous Free Metallic Cations Combining Hemodialysis and Chelating Polymer. Sci Rep 11, 19948 (2021).

### Pharmaceutical Composition of the Functionalized Polymer

The functionalized polymer for use is administered orally as such or may be formulated in the form with one or more pharmaceutically acceptable excipients.

Any suitable excipients known to those of ordinary skill in the art for use in pharmaceutical compositions may be employed in the compositions described herein.

In a specific embodiment, the pharmaceutical composition may be a liquid form suitable for oral administration, such as an aqueous solution of said functionalized polymer. In another specific embodiment, the pharmaceutical composition may be a solid dosage form suitable for oral administration. Solid dosage forms for oral administration include capsules, tablets, pills, powders, and granules. In a preferred embodiment, the pharmaceutical composition is a capsule or a tablet.

In an embodiment, the release of the capsule or tablet content may be immediate or modified such as delayed, targeted or extended. In a preferred embodiment the solid dosage form is an immediate release dosage form.

A second object of the disclosure pertains to an oral formulation comprising a functionalized polymer of the disclosure, and one or more pharmaceutically acceptable excipients.

In an embodiment, the pharmaceutically acceptable excipients comprises fillers, disintegrants, lubricants, glidants.

### Fillers

Fillers, (also referred to as a diluents, dilutants or thinners) are substance which are added to the drug substance in order to make the latter suitable for oral administration (e.g., capsules, tablets). Fillers themselves should not produce any pharmacological effect on human being. Examples of fillers include mannitol, microcrystalline cellulose, lactose monohydrate, anhydrous lactose, corn starch, xylitol, sorbitol, sucrose, dicalcium phosphate, maltodextrin, and gelatin.

### Disintegrants

Disintegrants are added to oral solid dosage forms to aid in their deaggregation. Disintegrants are formulated to cause a rapid break-up of solids dosage forms when they come into contact with moisture. Disintegration is typically viewed as the first step in the dissolution process. Examples of disintegrants include the modified starch such as sodium starch glycolate, sodium carboxymethyl starch, and pre-gelatinized starch, crosslinked polymers, such as crosslinked polyvinylpyrrolidone (crospovidone) or crosslinked sodium carboxymethyl cellulose (croscarmellose sodium), and calcium silicate.

### Lubricants

Lubricants are substances that we use in tablet and capsule formulations in order to reduce the friction. Lubricant can facilitate extrusion of tablets from matrix, thus preventing formation of scratches on their surfaces. By nature lubricants can be divided into two groups: a) fats and fat-like substances; b) powdery substance. Powdery substances are more applicable then the fat-like ones, because the latter impact on solubility and chemical stability of the tablets. Powdery lubricants are introduced by powdering of granulate. They provide constant-rate outflow of mass for tabletizing from hopper into matrix that guaranties accuracy and constancy of the drug substance dosage.

Examples of lubricants include magnesium stearate, hydrogenated castor oil, glyceryl behenate, calcium stearate, zinc stearate, mineral oil, silicone fluid, sodium lauryl sulfate, L-leucine, and sodium stearyl fumarate.

### Glidants

Glidants are blended with the formulation to enhance the tablet-core blend-material flow property. During the early stage of compression, glidants are mixed within the particle arrangement of the tablet powder blend to improve flowability and uniformity within the die cavity of tablet presses. Glidants encourage the flow of tablet granulation by diminishing friction between particles. The effect of glidants on the flow of the granules depends on the size and shape of the particles of the granules and the glidants. Above a certain concentration, the glidant will in fact function to inhibit flowability. In tablet manufacture, glidants are usually added just prior to compression. Examples of glidants include colloidal silicon dioxide, starch, magnesium stearate and talc.

Any suitable excipients known to those of ordinary skill in the art in pharmaceutical compositions may be further employed in the compositions described herein.

The unit dose of said functionalized polymer is of between 0.1 mg and 500 mg, preferably between 1 mg and 100 mg, more preferably between 2 mg and 10 mg, even more preferably about 5 mg.

### The Subject Population to be Preferably Targeted by the Treatment Methods

The treatment methods disclosed herein are suitable for subjects having a disease correlated with heavy metal-induced toxicity or heavy metal-induced carcinogenicity.

Without wishing bound by any theory, it is believed that the present method of treatment disclosed herein prevents the uptake of said heavy metal in the body of a subject.

In specific embodiments, said subject is a mammal, for example a human subject.

Accordingly, the methods of the present disclosure are particularly suitable for a subject exposed to heavy metal present in contaminated water and/or foodstuff and/or air pollution, for example from a contamination, a pollution, an accident, a combat zone such as dirty bomb.

Examples of diseases correlated with heavy metal-induced toxicity or heavy metal-induced carcinogenicity include without limitation, kidney diseases, liver diseases, gastro-intestinal diseases, cardiovascular diseases, respiratory disease, bone diseases, brain diseases, developmental abnormalities, neurologic and neurobehavioral disorders, diabetes, hearing loss, hematologic and immunologic disorders, and cancer disorders.

Said kidney disease may be particularly selected from the group consisting of chronic tubulointerstitial nephritis, end stage renal disease, Fanconi syndrome, chronic kidney disease.

Said liver disease may be particularly selected from the group consisting of nonalcoholic steatohepatitis (NASH), nonalcoholic fatty liver (NAFLD), hepatic fibrosis.

Said gastro-intestinal disease may be particularly selected from the group consisting of dysbiosis, inflammatory bowel diseases such as Crohn's disease or ulcerative colitis.

Said cardiovascular disease may be particularly selected from the group consisting of hypertension, stroke, atherosclerosis, peripheral vascular or arterial disease, coronary artery disease, congestive heart failure.

Said respiratory disease may be particularly selected from the group consisting of pneumonitis, pulmonary oedema, acute tracheobronchitis, pulmonary fibrosis, asthma, lung cancer.

Said bone disease may be particularly selected from the group consisting of osteomalacia, osteoarthritis, degenerative disk disease and osteoporosis.

Said brain disease may be particularly selected from the group consisting of dementia.

Said developmental abnormality may be particularly selected from the group consisting of congenital and neurological defects, developmental delays, and learning disabilities.

Said neurologic and neurobehavioral disorder may be particularly selected from the group consisting of autism spectrum disorders.

Said hematologic and immunologic disorder may be particularly selected from the group consisting of autoimmune diseases.

In specific embodiments, said disease is end stage renal disease or inflammatory bowel disease.

In a specific embodiment, such disease correlated with heavy metal-induced toxicity or heavy metal-induced carcinogenicity is due to the absorption of radioactive isotopes as listed herein of heavy metals in contaminated areas following nuclear incident or terrorism or war, thus contaminating, polluting water and/or air and/or foodstuff.

### The Methods of Treatment

The functionalized polymer as disclosed in the previous section, and more preferably the functionalized statistic chitosan, and their pharmaceutical compositions, are useful as a drug in methods for treating diseases correlated with heavy metal-induced toxicity or heavy metal-induced carcinogenicity in a subject in need thereof, and more particularly in the sub-population of subjects as defined above.

Said functionalized polymer is administered orally to the subject in an amount sufficient to prevent the uptake of said heavy metal in the body of a subject.

The pharmaceutical composition may be administered orally in any manner appropriate to the disease or disorder to be treated as determined by persons of ordinary skill in the medical arts.

An appropriate dose and a suitable duration and frequency of administration will be determined by such factors as discussed herein, including the condition of the subject, the type and severity of the subject's disease, the particular form of the active ingredient, and the method of administration.

In general, an appropriate dose (or effective dose) and treatment regimen provides the pharmaceutical composition in an amount sufficient to provide a therapeutic effect, for example, an improved clinical outcome, such as more frequent complete or partial remissions, or longer disease-free and/or overall survival, or a lessening of symptom severity or other benefit as described in detail herein.

In an embodiment, the functionalized polymer is administered once, twice or thrice a day to the subject in need thereof.

Typical daily dose may comprise between 0.1 mg and 500 mg, preferably between 1 mg and 100 mg, more preferably between 2 mg and 10 mg, even more preferably about 5 mg, preferably the functionalized statistic chitosan.

### FIGURES

FIG. 1: Qualitative biodistribution with fluorescence within Kidneys, Liver, Brain, Spleen, Heart, Urine, Lungs, Bone, Skin, Blood, Muscle, Stomach, Intestines, & Colon after 1, 2, 4, and 24 h (example 1).

FIG. 2: Average Percent of Administered Gd Within Digestive Tract Over 24 h (example 1).

FIG. 3: Body Weight Changes over 14-day, daily administration of MEX-CD1 in mice (example 2).

FIG. 4: Hematology after 14-day, daily administration of MEX-CD1 within Mice (example 2). Hematology assays were performed after the sacrifice of the mice. These results provide evidence of acute heavy metal poisoning within the saline-treated mice. All relations are non-significant, except those defined: * p<0.05, ** p<0.01, *** p<0.001, **** p<0.0001.

FIG. 5: Blood Concentrations of Lead and Cadmium after 14-day, daily administration of MEX-CD1 in mice (example 2). All mice were sacrificed after 15 days of exposure. NS p>0.05, * p<0.05, ** p<0.01, *** p<0.001.

FIG. 6: Graphic Representation of Chelating Abilities of MEX-CD1 for Lead and Cadmium in different concentrations (example 4). Technique used: HPLC-MS.

FIG. 6a: HPLC-MS chromatogram of the detection of lead interacting with MEX-CD1 (example 4).

FIG. 6b: HPLC-MS chromatogram of the detection of cadmium interacting with MEX-CD1 (example 4).

FIG. 7: Direct Evidence of Lead Chelation by MEX-CD1 using High Performance Liquid Chromatography coupled with Mass Spectrometry (HPLC-MS) (example 4).

FIG. 8: Graphic Representation of Chelating Ability of MEX-CD1 for Lead (example 4). Technique used: ICP-MS.

FIG. 9: Graphic Representation of Chelating Ability of MEX-CD1 for Lead and cadmium (example 4). Technique used: ICP-MS.

### EXAMPLES

Hereinafter, the present disclosure is described in more details and specifically with reference to the examples, which however are not intended to limit the present invention.

### Materials and Methods

### Preparation of Solutions:

### Solution 1: 10 g/L MEX-CD1

Lyophilized MEX-CD1 was dissolved in ultra-pure water at a concentration of 10 g/L.

### Solution 2: 10 g/L MEX-DTPA

Lyophilized MEX-DTPA was dissolved in ultra-pure water at a concentration of 10 g/L.

### Solution 3: 10 g/L MEX-DOTAM

Lyophilized MEX-DOTAM was dissolved in ultra-pure water at a concentration of 10 g/L.

### Solution 4: Biodistribution MEX-CD1 Mixture

Lyophilized MEX-CD1 obtained through the synthesis as described below and further marked with gadolinium was dissolved in ultra-pure water at a concentration of 10 g/L.

Lyophilized MEX-CD1 marked with Cyanine 5.5 was dissolved in ultra-pure water at a concentration of 10 g/L. 1 mL of each solution was combined to create the mixture solution containing 5 g/L of each marked MEX-CD1.

### Solution 6: 5 g/L MEX-CD1+3.5 g/L NaCl

3.5 g/L NaCl was dissolved in ultra-pure water. Lyophilized MEX-CD1 was then dissolved in this solution with a final concentration of 5 g/L. This solution was sterilized at 121° C. for 20 minutes.

### Solution A: Concentrated Salt Solution for In Vitro Laboratory Testing

263 g/L NaCl, 3.35 g/L KCl, 6.24 g/L CaCl2, 2.14 g/L MgCl2, 10.8 g/L Acetic Acid, 45 g/L Glucose in ultra-pure water

### Solution B: 0.1 M Acetate Buffer, pH 4.6

The acetate buffer contains 11.4 mL of acetic acid (MS grade), 15.4 g of ammonium acetate, and 2 L of ultra-pure water.

### Experimental Protocols:

### Protocol 1: High Pressure Liquid Chromatography-Mass Spectroscopy (HPLC-MS)

The eluent used for this method was acetate buffer (solution B, prepared with MS-Grade certified products), Ie injection volume was 10 uL, and the flow rate was fixed at 0.4 mL/min. The column used was a SEC Polysep GFC-P 4000 series. The HPLC is coupled to a Inductively Coupled Plasma Mass Spectrometer (ICP-MS) for the detection of chosen isotopes.

### Protocol 2: Vivaspin Ultrafiltration Experimentation

20 mL Vivaspin Centrifugation tubes with an internal membrane of 30 kDa were used. The centrifugation was performed at 4000 rpm for 10-30 minute cycles at a time. After each centrifugation, the solution that had passed through the membrane was collected for each sample, henceforth referred to as the undernatant. This was performed in one passage with ideally ~0.5 mL of supernatant remaining and collected for ICP-MS analysis.

The samples, including the original solution, the supernatant, and the undernatant, were analyzed by ICP-MS to determine the concentration of metals of interest within each solution. The samples were diluted by a factor of (at least) 10 using 1% HNO3. An internal standard (indium) was added to each sample with a final concentration of 2 ppb In. The analyses were performed in either KED (Kinetic Energy Discrimination) mode, which introduced a flux of helium within the chamber, or standard mode. KED was employed with the analysis was coupled with other analyses of more sensitive elements, but the majority of the samples were done in standard mode for: 208Pb, 206Pb, 111Cd, 112Cd, 114Cd.

A calibration curve ranging from 0.01 to 10 ppb for lead and cadmium was created to allow the system to convert the counts per second recorded for each sample into a concentration in ppb.

### Synthesis of Functionalized Polymer of the Invention:

### MEX-CD1 (Chitosan as Polymer and DOTAGA as Chelating Moiety)

In the first step, 60 g of chitosan, 4 L of ultra-pure water, and 45 mL of glacial acetic acid are introduced into a reactors of 10 L and agitated for 16 hours at a pH of 4.5±0.5. A solution of pale yellow is observed.

In the second step, 1.2 L of 1,2-propanediol is added to the pale yellow solution obtained from step 1 and agitated for 1 hour. A solution composed of 14 mL of acetic anhydride in 600 mL of 1,2-propanediol is slowly added over 10 minutes to obtain a homogeneous acetylation along the polymer chain. The medium is maintained and agitated for 4 hours.

The acetylation rate can be determined by elementary analysis. The non-acetylated monomer of the polymer (monomer B) presents a molar mass of 161.2 gmol⁻¹ (C₆NO₄H₁₁) while the acetylated monomer of the polymer (monomer A) presents a molar mass of 203.2 gmol⁻¹ (C₈NO₅H₁₃). The elementary analysis of the polysaccharide obtained after step 2 is the following: C 39.22%; H 7.55%; and N 6.77%, which corresponds to an acetylation rate of 29% (x=0.29) confirmed by ¹H NMR.

In the third step, 2 L of the solution obtained after the second step, is placed under agitation. 120 g of DOTAGA anhydride is added and agitated for 16 hours. At the end of this reaction, the solution is diluted by 10 in ultra-pure water and purified by tangential purification using a membrane of 100 kDa. After a first step of dilution ending with a volume of 16 L, the solution is filtered with 480 L 0.1 M acetic acid maintaining a constant volume of 16 L, then with 320 L of ultra-pure water. This purification ends with a re-concentration to a volume of 8 L. HPLC-UV allows to verify that the DOTAGA unreacted has been successfully removed from the solution. The solution with a concentration of 10 g/L is then filtered using a nylon filter (0.4 um) before lyophilisation.

¹H NMR allows to determine the percentage of monomers functionalized with DOTAGA (y) on the polymer known to have an acetylation percentage, x.

The quantity of grafted DOTAGA is determined by spectrophotometric UV dosage using at 295 nm. MEX-CD1 contains 0.345 mmol of DOTAGA per gram of polymer.

The molar fraction of N-acetyl glucosamine repeat units x is x=0.29 using 1H-NMR, the molar fraction of the DOTAGA-grafted repeat units y is y=0.075 using 1H-NMR and dosage by copper using HLPC-SEC.

### MEX-DTPA (Chitosan as Polymer and DTPA as Chelating Moiety)

In the first step, 1 g of chitosan, 66 mL of ultra-pure water, and 0.84 mL of glacial acetic acid are introduced and agitated for 16 hours at a pH of 4.5±0.5. A solution of pale yellow is observed.

In the second step, 20 mL of 1,2-propanediol is added to the pale yellow solution obtained from step 1 and agitated for 1 hour. A solution composed of 0.704 mL of acetic anhydride in 30 mL of 1,2-propanediol is slowly added over 10 minutes to obtain a homogeneous acetylation along the polymer chain. The medium is maintained and agitated for 4 hours.

In the third step, 0.8 g of DTPA bis-anhydride is added to the previously solution and agitated for 16 hours. At the end of this reaction, the solution is diluted by 10 in ultra-pure water and purified by tangential purification using a membrane of 100 kDa. After a first step of dilution ending with a volume of 1 L, the solution is filtered with 5 L 0.1 M acetic acid maintaining a constant volume of 1 L, then with 5 L of ultra-pure water. This purification ends with a re-concentration to a volume of ~100 mL. HPLC-UV allows to verify that the DTPA unreacted has been successfully removed from the solution.

The quantity of grafted DTPA is determined by spectrophotometric UV dosage using at 295 nm. MEX-DTPA contains 0.21 mmol of DTPA per gram of polymer.

The molar fraction of N-acetyl glucosamine repeats units x is x=0.58 using 1H-NMR, the molar fraction of the DTPA-grafted repeat units y is y=0.045 using 1H-NMR.

The MEX-DTPA as herein synthesized is useful for the treatment method as described in the present disclosure.

### MEX-DOTAM (Chitosan as Polymer and DOTAM as Chelating Moiety)

In the first step, 1 g of chitosan, 66 mL of ultra-pure water, and 0.84 mL of glacial acetic acid are introduced and agitated for 16 hours at a pH of 4.5±0.5. A solution of pale yellow is observed.

In the second step, 20 mL of 1,2-propanediol is added to the pale yellow solution obtained from step 1 and agitated for 1 hour. A solution composed of 0.704 mL of acetic anhydride in 30 mL of 1,2-propanediol is slowly added over 10 minutes to obtain a homogeneous acetylation along the polymer chain. The medium is maintained and agitated for 4 hours. This solution is then purified by tangential purification using a membrane of 100 kDa, and 7 L ultra-pure water to remove the solvents and re-concentrated to ~100 mL. Using NaOH, the pH of this solution is raised to 7±0.1. 8 mL of DMSO is added to the solution for an environment of 5% DMSO in water and agitated for 1 hour.

In the third step, 11 mL of a 100 g/L solution of DOTAM NHS-ester in DMSO is added over 10 minutes. Solution then diluted ×2 with ultra-pure water and agitated for 16 hours. At the end of this reaction, the solution is diluted by 10 in ultra-pure water and purified by tangential purification using a membrane of 100 kDa. After a first step of dilution ending with a volume of 1 L, the solution is filtered with 5 L 0.1 M acetic acid maintaining a constant volume of 1 L, then with 5 L of ultra-pure water. This purification ends with a re-concentration to a volume of ~100 mL. HPLC-UV allows to verify that the DOTAM unreacted has been successfully removed from the solution.

The quantity of grafted DOTAM is determined by spectrophotometric UV dosage using at 295 nm. MEX-DOTAM contains 0.049 mmol of DOTAM per gram of polymer.

The molar fraction of N-acetyl glucosamine repeats units x is x=0.58 using 1H-NMR, the molar fraction of the DOTAGA-grafted repeat units y is y=0.0097 using 1H-NMR.

The MEX-DOTAM as herein synthesized is useful for the treatment method as described in the present disclosure.

### PEG-DTPA (PEG as Polymer and DTPA as Chelating Moiety)

The PEG used is an 8-arm PEG amine with a molecule weight of 40 kDa according to the following formula (V)

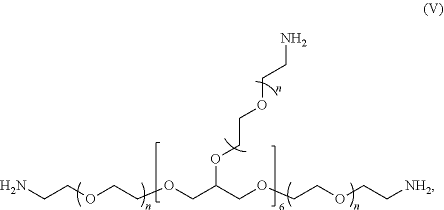

wherein n is 111.3, purchased from Creative PEGWork. The DTPA bis-anhydride was provided by Chematech. The synthesis was performed in DMSO (Fischer Chemicals). The cassettes VIVAFLOW 30 kDa used for purification were purchased from Sartorius.

0.36 g of DTPA bis-anhydride was dissolved in 10.26 mL DMSO (35 g/L) under agitation at 40° C. for 1 hour. In the mean-time, a mass of 0.27 g of PEG was weighed and dissolved in 43.36 mL DMSO. This PEG solution was then added directly into the DTPA solution and agitated at 40° C. for 1.5 hours. The solution was the purified using ultra-pure water and 10⁻² M HCl with the VIVAFLOW 30 kDa cassette for a final purification factor ~10000.

The quantity of grafted DTPA is determined by spectrophotometric UV dosage using at 295 nm. PEG-DTPA contains 0.18 mmol of DTPA per gram of polymer. The molar fraction of the DTPA-grafted repeat units y is y=1 using 1H-NMR.

The structure of the PEG-DTPA synthetized has the following formula (VI):

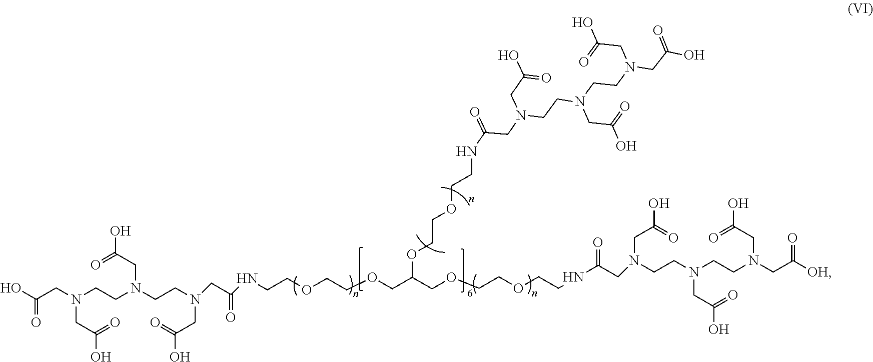

wherein n is 111.3.

The PEG-DTPA as herein synthesized is useful for the treatment method as described in the present disclosure.

### PEG-DOTAM (PEG as Polymer and DOTAM as Chelating Moiety)

The PEG used is an 8-arm PEG amine with a molecule weight of 40 kDa, purchased from Creative PEGWork. The DOTAM NHS-ester was provided by Chematech as a solution of 100 g/L in DMSO. The synthesis was performed in DMSO (Fischer Chemicals). The cassettes VIVAFLOW 30 kDa used for purification were purchased from Sartorius.

3.5 mL of the 100 g/L DOTAM NHS-ester/DMSO solution was added to a round-bottom flask under agitation at 40° C. for 1 hour. In the meantime, a mass of 0.27 g of PEG was weighed and dissolved in 43.36 mL DMSO. This PEG solution was then added directly into the DTPA solution and agitated at 40° C. for 1.5 hours. The solution was the purified using ultra-pure water and 0.1 M acetic acid with the VIVAFLOW 30 kDa cassette for a final purification factor ~10000.

The quantity of grafted DOTAM is determined by spectrophotometric UV dosage using at 295 nm. PEG-DOTAM contains 0.036 mmol of DOTAM per gram of polymer. The molar fraction of the DOTAM grafted on amine function of ethylene glycol monomers y is y=0.21 using 1H-NMR.

The structure of the PEG-DOTAM synthetized has the following formula (VII):

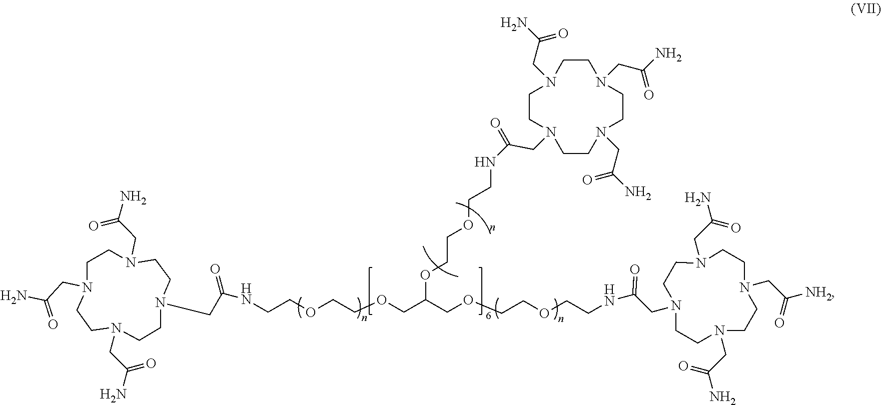

wherein n is 111.3.

The PEG-DOTAM as herein synthesized is useful for the treatment method as described in the present disclosure.

### Example 1: Biodistribution of Orally Administered MEX-CD1 in Mice

0.2 mL of solution 4 was orally administered for 8 mice. 2 mice were sacrificed at each time point: 1, 2, 4, and 24 hours. The fluorescence study was performed using a CCD camera and the following parameters: 2 sets of spotlights for excitation at 633 and 470 nm and 2 sets of filters at 680±20 and 520±20 nm, then the organs were digested in nitric acid for gadolinium quantification using ICPMS.

### Results

Qualitative Biodistribution with Fluorescence

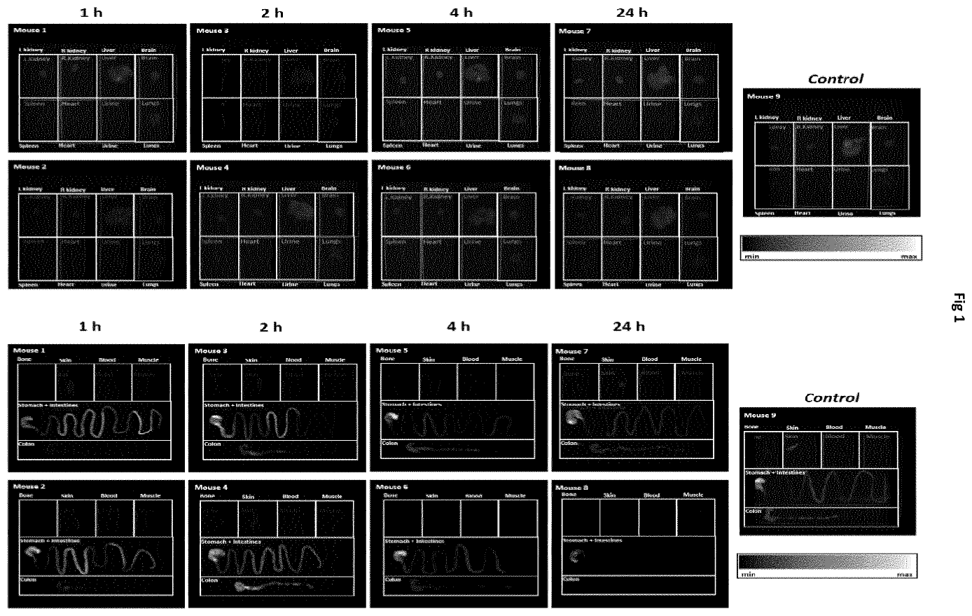

As shown in FIG. 1, no fluorescence is observed at any time point within Kidneys, Liver, Brain, Spleen, Heart, Urine, Lungs, Bone, Skin, Blood, & Muscle after 1, 2, 4, and 24 h. Fluorescence is only observed within the Stomach, Intestines, and Colon after 1, 2, and 4 hours. A small level of autofluorescence is seen within the stomach of the control mouse.

The fluorescence results show the presence of MEX-CD1 within only the stomach, intestines, and colon (digestive tract). Some auto-fluorescence is observed within the stomach of the control mouse which explains the remaining fluorescence at 24 hours within the two stomachs.

Quantitative Biodistribution with ICP-MS

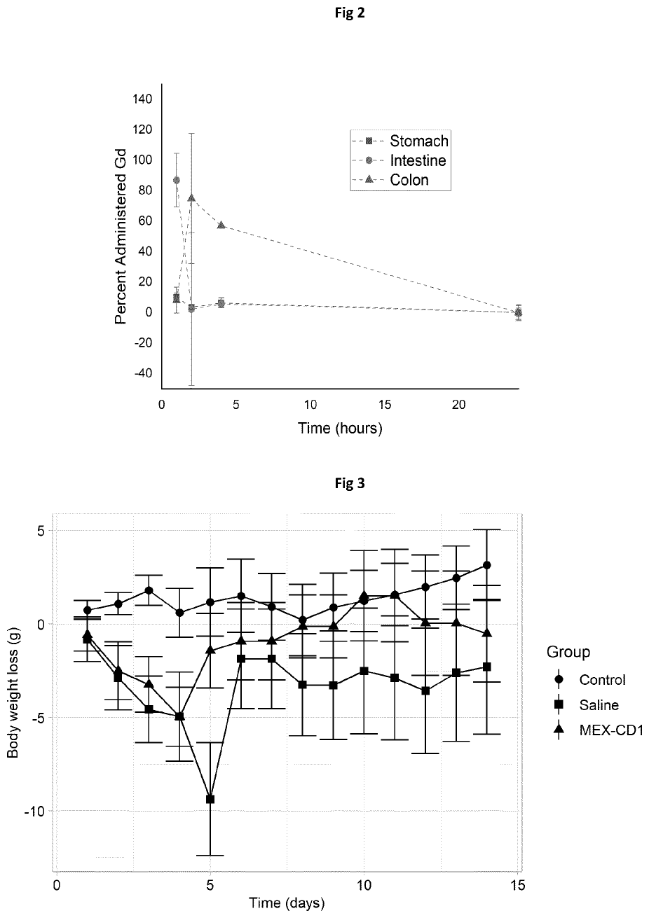

As shown in FIG. 2, each time point had two experimental mice, therefore the average percent gadolinium was taken. This was then plotted with the standard deviation in FIG. 1. The trend shows that within the first hour, the majority of MEX-CD1 is already found within the intestine and at 24 hours, all of the MEX-CD1 is eliminated from the digestive system of the mice.

Other organs were also assessed, in which all presented less than 1% of the administered gadolinium in concentration and was therefore considered as zero.

Therefore, orally administered MEX-CD1 remains within the digestive tract, does not pass the intestinal membrane into the blood, and is completely eliminated after one day.

### Example 2: Orally Administered MEX-CD1 in Mice Exposed to Heavy Metals

The study has been performed as described below:
Control group—fed normal rodent feed & water with no treatment
Saline group—fed contaminated rodent feed, normal water, oral administration of saline solution once daily for 14 days
MEX-CD1 group—fed contaminated rodent feed, normal water, oral administration of MEX-CD1 solution once daily for 14 days

$$Contaminated rodent feed = 7 ppm ( mg / kg ) Cd + 50 ppm ( mg / kg ) Pb$$

$$Oral administration = 0.2 mL 3.5 NaCl in H2O or$$

$$0.2 mL of 5 g / L MEX - CD 1 + 3.5 NaCl in H2O$$

### Results

### Body Weight

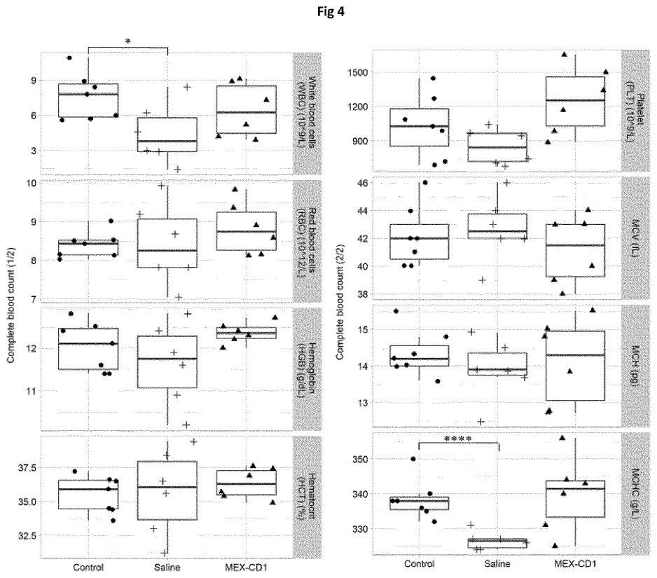

As shown in FIG. 3, the decrease in body weight observed in the saline- and MEX-CD1-treated groups can be attributed to the exposure to contaminated rodent feed. The groups then stabilize in body weight after 6 days. This proves that the oral administration of MEX-CD1 does not affect the health of the mice, as portrayed by change in body weight over time.

### Liver Enzyme Activity

TABLE 1
Liver Enzyme Activity after 14-day, daily Oral
Administration of Saline or MEX-CD1 within Mice

| | ALP, U/L | | ALT, U/L | | AST, U/L | |
|---|---|---|---|---|---|---|
| Group | (mean ± SD) | p value | (mean ± SD) | p value | (mean ± SD) | p value |
| Control | 259 ± 83 | | 79 ± 55 | | 200 ± 88 | |
| Saline | 345 ± 183 | 0.4751 | 65 ± 6 | 0.5698 | 201 ± 47 | 0.9777 |
| MEX-CD1 | 201 ± 35 | 0.2912 | 139 ± 136 | 0.3914 | 225 ± 78 | 0.5924 |

The enzyme activity levels of the mice remained non-statistically significant in all groups, providing evidence that the oral administration of MEX-CD1 does not impact the liver function.

### Hematology

As shown in FIG. 4, acute heavy metal poisoning is evidenced in some hematological parameters among the saline-treated mice (specifically White Blood Cell (WBC) and MCHC, which are significantly different, as well as slight changes in Platelets (PLT) and MCH). This onset of acute heavy metal poisoning occurs in a similar fashion as to observations previously described in literature. This poisoning is reverted to normal levels with the oral administration of MEX-CD1. Not only does this provide evidence that there is no negative impact of this treatment on haematological parameters, it also shows the beneficial impact of this orally administered therapy to protect against the exposure to heavy metals.

### Blood Concentrations of Lead and Cadmium

As shown in FIG. 5, there is a significant increase in blood lead and cadmium concentration when mice are exposed to contaminated rodent food. MEX-CD1, in the case of lead, helps alleviate the concentration found within the blood of the mice by oral administration once per day. MEX-CD1 shows minimal effect in the cadmium concentration due its trace level.

### Example 3: In Vitro Chelation of Lead and Cadmium in Complex Environments by MEX-Polymers

### Polymer 1—MEX-CD1

The samples were creating using solution A as defined in the materials and methods part. Using the metal standards purchased from SCP Science with concentrations of 1000 ppm of Pb²⁺ or Cd²⁺ in 5% HNO3 as well as solution 1, 4 samples were created. The pH was adjusted using 1 M NaOH. Following protocol 2, the samples were experimentally analyzed.

TABLE 2

| | Base | | Concentration | |
|---|---|---|---|---|
| | Solution | Concentration | of Pb & Cd | |
| Sample | Used | MEX-CD1 | (ppb) | pH |
| 1 | Solution A | 0 | 100 | 2 |
| 2 | Solution A | 0.1 g/L | 100 | 2 |
| 3 | Solution A | 1 g/L | 50 | 4 |
| 4 | Solution A | 1 g/L | 50 | 5 |

TABLE 3

| | Pb²⁺ | | | Cd²⁺ | | |
|---|---|---|---|---|---|---|
| | (ppb) | | | (ppb) | | |
| Sample | Original | Supernatant | Undernatant | Original | Supernatant | Undernatant |
| 2 | 86.7 | 123.9 | 85.0 | 70.2 | 105.5 | 74.9 |
| 3 | 50.3 | 751.4 | 21.3 | 36.2 | 140.9 | 33.0 |
| 4 | 45.4 | 314.9 | 31.2 | 31.1 | 218.5 | 24.3 |

### Polymer M2E—MEX-DTPA

The samples were prepared using solution A. Using the metal standards purchased from SOP Science with concentrations of 1000 ppm of Pb2+ or Cd2+ in 5% HN3 as well as solution 2, 7 samples were created. The pH was adjusted using 1 M NaOH. Following protocol 2, the samples were experimentally analyzed.

TABLE 4

| | Base | | Concentration | |
|---|---|---|---|---|
| | Solution | Concentration | of Pb & Cd | |
| Sample | Used | MEX-DTPA | (ppb) | pH |
| 1.1 | Solution A | 0 | 100 | 2 |
| 1.2 | Solution A | 0 | 50 | 2 |
| 2 | Solution A | 0.1 g/L | 100 | 2 |
| 3 | Solution A | 1 g/L | 50 | 2 |
| 4 | Solution A | 1 g/L | 50 | 3 |
| 5 | Solution A | 1 g/L | 50 | 4 |
| 6 | Solution A | 1 g/L | 50 | 5 |

TABLE 5

| | Pb²⁺ | | | Cd²⁺ | | |
|---|---|---|---|---|---|---|
| | (ppb) | | | (ppb) | | |
| Sample | Original | Supernatant | Undernatant | Original | Supernatant | Undernatant |
| 2 | 83.7 | 126.2 | 85.7 | 67.5 | 106.4 | 72.0 |
| 3 | 49.7 | 70.2 | 52.4 | 35.4 | 37.8 | 39.5 |
| 4 | 48.6 | 241.2 | 42.9 | 34.4 | 113.2 | 34.9 |
| 5 | 44.6 | 1027.8 | 12.6 | 32.4 | 751.1 | 10.5 |
| 6 | 45.3 | 840.7 | 17.5 | 32.5 | 519.8 | 17.0 |

### Polymer 3—MEX-DOTAM

The samples were prepared using solution A. Using the metal standards purchased from SOP Science with concentrations of 1000 ppm of Pb2+ or Cd2+ in 5% HN3 as well as solution 3, 7 samples were created. The pH was adjusted using 1 M NaOH. Following protocol 2, the samples were experimentally analyzed.

TABLE 6

| | Base | | Concentration | |
|---|---|---|---|---|
| | Solution | Concentration | of Pb & Cd | |
| Sample | Used | MEX-DOTAM | (ppb) | pH |
| 1.1 | Solution A | 0 | 100 | 2 |
| 1.2 | Solution A | 0 | 50 | 2 |
| 2 | Solution A | 1 g/L | 50 | 2 |
| 3 | Solution A | 1 g/L | 50 | 3 |
| 4 | Solution A | 1 g/L | 50 | 4 |
| 5 | Solution A | 1 g/L | 50 | 5 |

TABLE 7

| | Pb²⁺ | | | Cd²⁺ | | |
|---|---|---|---|---|---|---|
| | (ppb) | | | (ppb) | | |
| Sample | Original | Supernatant | Undernatant | Original | Supernatant | Undernatant |
| 2 | 44.0 | 70.1 | 46.3 | 33.1 | 23.6 | 40.9 |
| 3 | 41.2 | 113.3 | 29.1 | 33.3 | 33.0 | 35.7 |
| 4 | 40.2 | 100.1 | 26.3 | 31.8 | 71.2 | 17.5 |
| 5 | 42.3 | 271.3 | 15.6 | 30.5 | 271.8 | 2.8 |

Since the original solution was centrifuged and passed through the membrane in one step, the original concentration and undernatant concentration are comparable. For each polymer, efficient chelation of cadmium and lead is observed at pH 4 and 5, exhibited by a lower concentration of metal within the undernatant compared to the original. Minimal lead chelation is observed with MEX-DOTAM and MEX-DTPA at pH 2 and 3, while efficient chelation is observed at pH 4 and 5. Due to the more efficient chelation capacities of MEX-DTPA and MEX-DOTAM within low pH environments in comparison with MEX-CD1, these polymers could display similar or improved chelation capacities in vivo compared to MEX-CD1.

### Example 4: In Vitro Chelation of Lead and Cadmium by MEX-CD1 in Simple & Complex Environments

### MEX-CD1 Chelation in Water

Using a metal mixture purchased from SCP Science of Pb and Cd, both with a concentration of 10 ppm in 5% HNO3, as well as solution 1, the following 6 samples were prepared in ultra-pure water, and analyzed by HPLC-MS following Protocol 1.

TABLE 8

| | | Concentration |
|---|---|---|
| | | en métaux |
| Echantillon | Polymère | (ppb) |
| 1 | 0.1 g/L MEX-CD1 | 0 |
| 2 | | 0.1 |
| 3 | | 0.5 |
| 4 | | 1 |
| 5 | | 5 |
| 6 | | 10 |

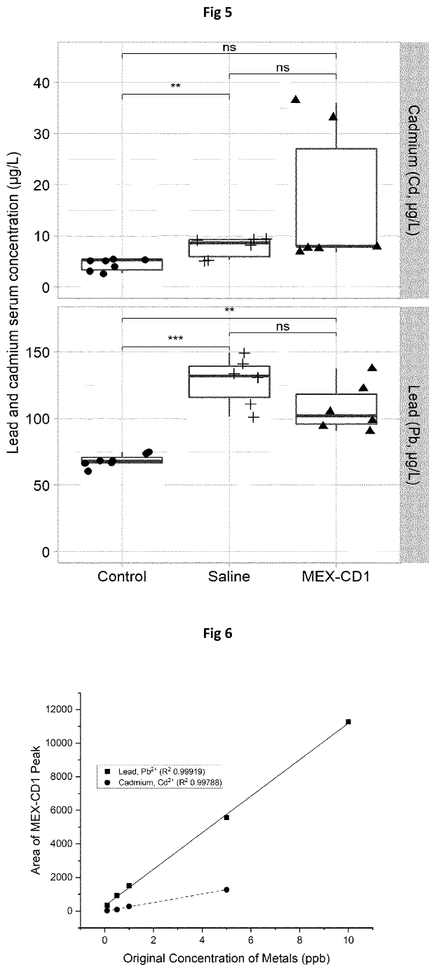

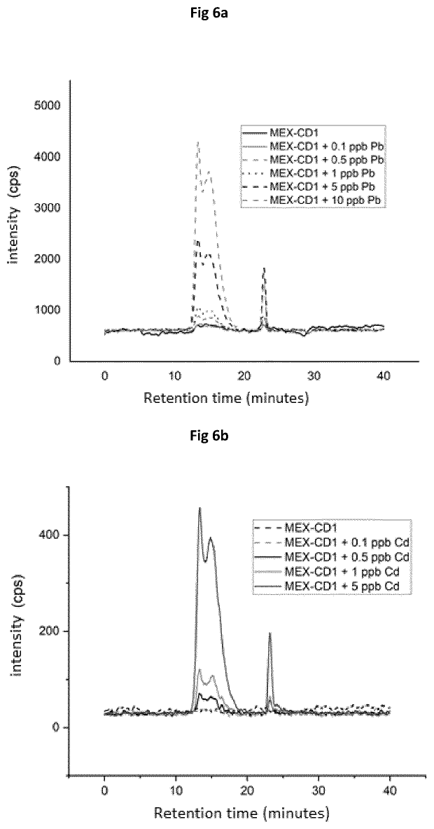

The FIGS. 6, 6a and 6b shows that the polymer at a concentration of 0.1 g/L within a concentrated solution composed of salts and acids is efficient to chelate lead. With increasing metal concentration, a larger polymer peak is observed at 11 minutes in FIGS. 6a and 6b corresponding to more chelation of these metals on the polymer. The linear relationship can be seen in FIG. 6 providing chelation evidence of these metals by MEX-CD1 within the desired concentration range.

### MEX-CD1 Chelation in Solution A

77 ppb of Pb²⁺ were added to solution A. This doped solution, as well as solution 1, solution B, and ultra-pure water were used to create different acidic environments. The chelation was tested within these different environments: concentrated acid (pH 2), pH 5, and pH 7.4. The pH was adjusted with NaOH and the samples were analyzed by HPLC-MS following Protocol 1.

TABLE 9

| | Dilution | |
|---|---|---|
| | Factor of | |
| Sample | Solution A | pH |
| Concentrated Solution | 1 | 2 |
| A | | |
| Concentration | 1 | 2 |
| Solution A + 0.1 g/L | | |
| MEX-CD1 | | |
| Solution A + | 5 | 5 |
| 0.1 g/L MEX-CD1 | | |
| Solution A + | 5 | 7.4 |
| 0.1 g/L MEX-CD1 | | |

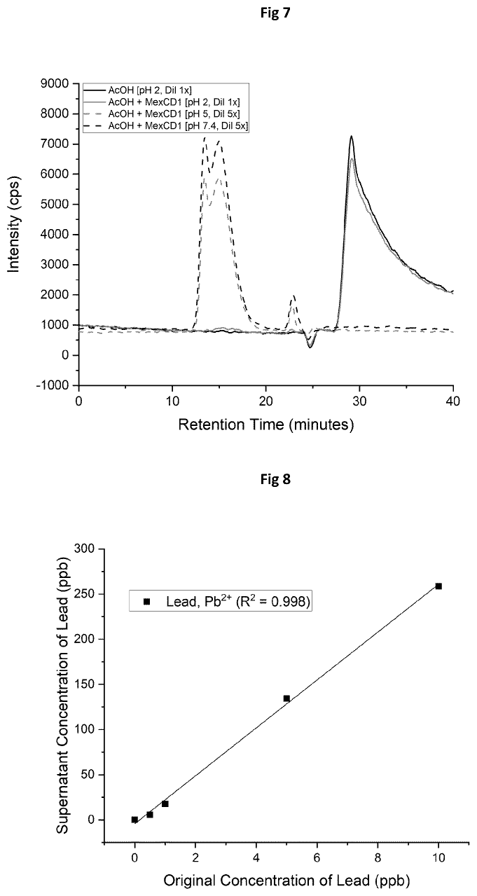

The FIG. 7 above shows that the polymer at a concentration of 0.1 g/L within a concentrated solution composed of salts and acids is efficient to chelate lead. At pH 5 and 7.4, a large portion of the lead is chelated and associated to the polymer which is represented by the chromatogram of HPLC/SEC-ICP-MS (lead detection) with an increase in the peak area at 15 minutes, specifically corresponding to the polymer.

### MEX-CD1 in Water

The samples were prepared using ultra-pure water, solution 1, and a solution of Pb²⁺ with a concentration of 50000 ppm in 5% HNO₃. Following Protocol 2, the samples were experimentally analyzed.

TABLE 10

| | | | | Results | | |
|---|---|---|---|---|---|---|
| Sample Preparation | | | | Original | Supernatant | Undernatant |
| | MEX-CD1 | Pb²⁺ | | [Pb²⁺] | [Pb²⁺] | [Pb²⁺] |
| Sample | concentration | (ppb) | pH | (ppb) | (ppb) | (ppb) |
| 1 | 0 | 10 | 6.40 | 5.418 | | 0.855 |
| 2 | 10 mg/L | 0 | 5.79 | 0.024 | 0.048 | 0.013 |
| 3 | | 0.1 | 5.73 | 0.113 | 0.543 | 0.019 |
| 4 | | 5 | 6.03 | 4.412 | 22.65 | 0.103 |
| 5 | | 10 | 5.78 | 8.841 | 50.25 | 0.102 |

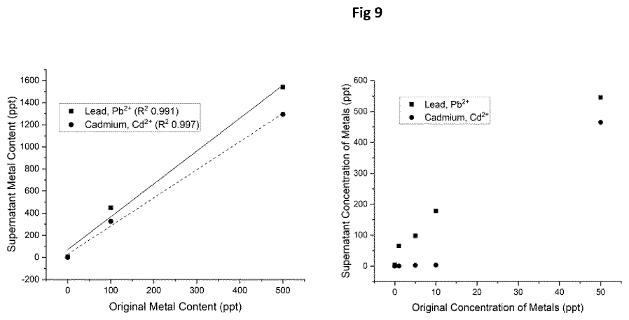

The FIG. 8 above shows the linear relationship between increasing lead concentration and the chelation by MEX-CD1.

### MEX-CD1 in Water, Ultra-Trace Chelation

A metal mixture solution containing 10 ppm of both Pb and Cd in 5% HNO3, purchased from SCP Science, was used with solution 1 and ultra-pure water to prepare the samples. Protocol 2 was used to experimentally analyze these samples.

TABLE 11

| | | Metal | |
|---|---|---|---|
| | MEX-CD1 | Concentration | |
| Sample | concentration | (ppt) | pH |
| 1 | 0 | 100 | ~6.5 |
| 2 | 1 mg/L | 0 | |
| 3 | | 1 | |
| 4 | | 5 | |
| 5 | | 10 | |
| 6 | | 50 | |
| 7 | | 100 | |
| 8 | | 500 | |
| 9 | | 1000 | |

FIG. 9 evidenced a chelation efficiency starting at 0.05 ppb (50 ppt) for lead and cadmium by ICP-MS analysis. Even in the domain of 0.01 ppb (10 ppt), lead shows an efficient chelation.

1. A method of using a functionalized polymer, for treating a disease correlated with heavy metal-induced toxicity or heavy metal-induced carcinogenicity in a subject in need thereof, wherein said functionalized polymer is soluble in an aqueous solution, wherein said functionalized polymer has an average molecular mass between kDa and 5000 kDa, wherein said functionalized polymer is comprised of monomeric units, wherein at least one of the monomeric units are functionalized with a chelating moiety, and wherein a therapeutically efficient amount of said functionalized polymer is administered orally to the subject.

2. The method of using the functionalized polymer of claim 1 wherein said subject in need thereof is a subject exposed to heavy metals present in contaminated water and/or foodstuff and/or air pollution.

3. The method of using the functionalized polymer of claim 1, wherein said functionalized polymer comprises at least 1 w % of the chelating moiety.

4. The method of using the functionalized polymer of claim 1, wherein said chelating moiety is selected from the group consisting of DOTA, NOTA, NODAGA, DOTAGA, DOTAM, NOTAM, DOTP, NOTP, TETA, TETAM, DTPA, Bz-DFO, DFO and mixtures thereof.

5. The method of using the functionalized polymer claim 1, wherein said disease correlated with heavy metal-induced toxicity or heavy metal-induced carcinogenicity is selected from the group consisting of kidney diseases, liver diseases, gastro-intestinal diseases, cardiovascular diseases, respiratory disease, bone diseases, brain diseases, developmental abnormalities, neurologic and neurobehavioral disorders, diabetes, hearing loss, hematologic disorders, immunologic disorders, and cancer disorders.

6. The method of using the functionalized polymer of claim 5, wherein:
a. said kidney disease is selected from the group consisting of chronic tubulointerstitial nephritis, end stage renal disease, Fanconi syndrome, and chronic kidney disease;
b. said liver disease is selected from the group consisting of nonalcoholic steatohepatitis (NASH), nonalcoholic fatty liver (NAFLD), and hepatic fibrosis;
c. said gastro-intestinal disease is selected from the group consisting of dysbiosis, and inflammatory bowel disease;
d. said cardiovascular disease is selected from the group consisting of hypertension, stroke, atherosclerosis, peripheral vascular or arterial disease, coronary artery disease, and congestive heart failure;
e. said respiratory disease is selected from the group consisting of pneumonitis, pulmonary oedema, acute tracheobronchitis, pulmonary fibrosis, asthma, and lung cancer;
f. said bone disease is selected from the group consisting of osteomalacia, osteoarthritis, degenerative disk disease and osteoporosis;
g. said brain disease is dementia;
h. said developmental abnormality is selected from the group consisting of congenital defects, neurological defects, developmental delays, and learning disabilities;
i. said neurologic and neurobehavioral disorder is a autism spectrum disorder; and
j. said hematologic and immunologic disorder is selected from the group consisting of autoimmune diseases.

7. The method of using the functionalized polymer of claim 1, wherein said heavy metal is selected from the group consisting of lead (Pb), cadmium (Cd), arsenic (As), chromium (Cr), mercury (Hg), cobalt (Co), aluminium (Al), antinomy (Sb), barium (Ba), bismuth (Bi), gallium (Ga), germanium (Ge), gold (Au), indium (In), nickel (Ni), platinum (Pt), silver (Ag), strontium (Sr), tellurium (Te), thallium (Tl), tin (Sn), titanium (Ti), vanadium (V), plutonium (Pu), cesium (Cs), cerium (Ce), zirconium (Zr), ruthenium (Ru), technetium (Tc), radium (Ra), thorium (Th), americium (Am), iridium (Ir), californium (Cf), polonium (Po) and uranium (U), or mixtures thereof.

8. The method of using the functionalized polymer claim 1, wherein the functionalized polymer is administered to the subject at a unit dose of between 0.1 mg and 500 mg.

9. The method of using the functionalized polymer of claim 1, wherein the functionalized polymer is a polysaccharides, wherein at least one of the monomeric units is functionalized with a chelating moiety.

10. The method of using the functionalized polymer of claim 1, wherein said functionalized polymer is a functionalized statistic chitosan with an average molecular mass between 100 kDa and 1000 kDa and wherein said functionalized polymer is a compound of formula (I): 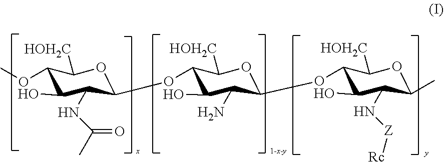
wherein
each Rc is the chelating moiety,
each Z is independently a linker which is a single bond or a hydrocarbon chain containing between 1 and 12 carbon atoms, said hydrocarbon chain is linear or branched and optionally wherein said hydrocarbon chain is an unsaturated hydrocarbon and contains one or more heteroatoms, wherein the heteroatom is optionally nitrogen, oxygen, sulfur or a halogen,
each x is between 0.005 and 0.7, and
each y is between 0.005 and 0.5,
wherein the ratio of y/x is greater than or equal to 0.01, and
the sum x+y being greater than or equal to 0.15.

11. The method of using the functionalized polymer for use according to claim 10, wherein said functionalized polymer is a compound of formula (III): 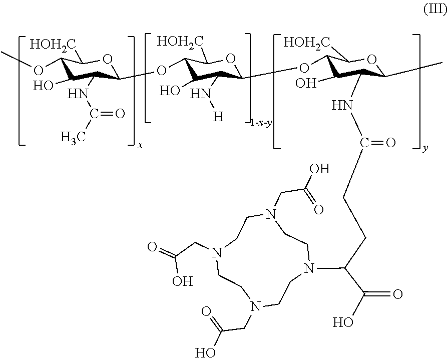
wherein x is between 0.25 and 0.4 and y is between 0.05 and 0.2.

12. The method of using the functionalized polymer of claim 1, wherein said functionalized polymer is formulated as a solid dosage form.

13. The method of using the functionalized polymer of claim 1, wherein the functionalized polymer is administered once, twice or thrice a day.

14. The method of using the functionalized polymer of claim 1, comprising administering the functionalized polymer and one or more pharmaceutically acceptable excipients in an oral formulation, wherein the unit dose of said functionalized polymer in the oral formulation is of between 0.1 mg and 500 mg.

15. The method of using the functionalized polymer of claim 14, wherein the oral formulation comprises a compound of formula (III): 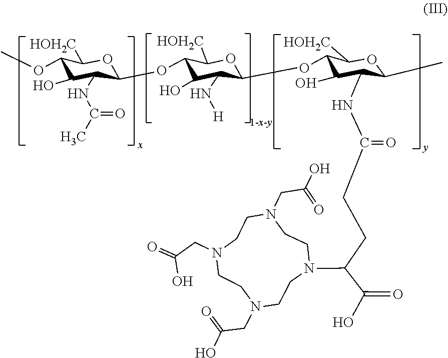
wherein x is between 0.25 and 0.4, and y is between 0.05 and 0.2.

16. The method of using the functionalized polymer of claim 1, wherein said functionalized polymer comprises between 1 w % and 40 w % of the chelating moiety.

17. The method of using the functionalized polymer of claim 6, wherein said inflammatory bowel disease is crohn's disease or ulcerative colitis.

18. The method of using the functionalized polymer of claim 1, wherein said heavy metal is selected from the group consisting of lead and cadmium.

19. The method of using the functionalized polymer of claim 1, wherein the functionalized polymer is administered to the subject at a unit dose of about 5 mg.

20. The method of using the functionalized polymer of claim 1, wherein the polysaccharide is chitosan, polyethylene glycol (PEG), polyvinyl alcohol (PVA), polyacrylic acid (PAA), or polyallylamine (PAH), wherein at least one of the monomeric unit is functionalized with a chelating moiety.
